(19) United States
(12) Patent Application Publication
(10) Pub. No.: US 2004/0147469 A1
(43) Pub. Date: Jul. 29, 2004

(54) Methods of inhibiting glial scar formation

(75) Inventors: Barbara Grimpe, Cleveland, OH (US); Jerry Silver, Cleveland, OH (US)

Correspondence Address: ROPES & GRAY LLP, ONE INTERNATIONAL PLACE, BOSTON, MA, 02110-2624, US

(73) Assignee: Case Western Reserve University, Cleveland, OH (US)

(21) Appl. No.: 10/698,190

(22) Filed: Oct. 31, 2003

Related U.S. Application Data
(60) Provisional application No. 60/423,082, filed on Nov. 1, 2002.
(60) Provisional application No. 60/471,447, filed on May 16, 2003.

(51) Int. Cl. A61K 48/00

(52) U.S. Cl. 514/44

(57) ABSTRACT

The present invention provides methods and compositions for inhibiting glial scar formation, methods and compositions for decreasing GAG content, methods and composition for decreasing proteoglycan gene expression, and methods and compositions for promoting neuronal regeneration. The present invention further provides methods of identifying additional agents that inhibit glial scar formation and/or promote neuronal regeneration.

### BACKGROUND OF THE INVENTION

One of the greatest obstacles in developing effective treatment for injuries and degenerative diseases of the nervous system is the poor regenerative capacity of neuronal cells. Regeneration of neuronal cells in the central nervous system is currently not possible, and although neuronal cells in the peripheral nervous system show some regenerative capacity, this occurs very slowly across limited nerve gaps. Thus, individuals who experience reduction in neuronal function following injury or diseases of the central nervous system typically see little or no improvement in their condition over time, while individuals experiencing reduction in neuronal function following injury or disease of the peripheral nervous system may experience some improvement, but only after a considerable period of time. Accordingly, there exists a need for improved methods and compositions to promote neuronal regeneration in the central and peripheral nervous systems.

Neurons have cellular processes called axons that migrate and establish connections with their targets. One of the results of damage to the nervous system is the formation of glial scar tissue. Glial scar tissue is composed of astrocytes, oligodendrocytes and microglia, as well as a rich meshwork of extracellular matrix proteins including proteoglycans. Glial scar tissue formed in response to cellular damage presents a physical and/or a molecular barrier to regeneration.

Proteoglycans are molecules consisting of one or more glycosaminoglycan (GAG) chains attached to a core protein. Glial scar tissue is rich in proteoglycans modified with various types of GAG moieties, and GAG modified proteoglycans have been shown in a variety of in vitro and in vivo systems to exert an inhibitory effect on regenerating neurons (reviewed in Silver (1994) J Neurol. 242: S22-4; Yu and Bellamkonda (2001) J. Neurosci. Res. 66: 303-310). These studies indicate that GAGs play an important role in the formation of and the inhibitory effects exerted by glial scar tissue (Margolis and Margolis (1997) Cell Tissue Res. 290: 343-8; Friedlander et al. (1994) J. Cell Biol. 125: 669-680).

Given the high concentration of GAG modified proteoglycans in glial scar tissue following injury or disease, there exists a need for methods and compositions that reduce glial scar formation following neuronal injury or disease, as well as a need for methods and compositions that reduce GAG modified proteoglycan content in neuronal tissue following injury or disease. The present invention aims to provide such methods and compositions.

Furthermore, given the role of GAG modified proteoglycans in inhibiting neuronal regeneration, there exists a need for methods and compositions for promoting neuronal regeneration following injury or disease. The present invention aims to provide such methods and compositions.

Given the enormous impact of injuries and diseases of the nervous system on the quality of life of affected individuals, and given that there currently exist no effective methods for promoting neuronal regeneration and thereby alleviating the negative effects of these injuries and diseases, methods and compositions which aim to enhance neuronal regeneration address an important and currently unmet need.

### SUMMARY OF THE INVENTION

The methods and compositions described in the present application address the need in the art for improved methods of promoting neuronal regeneration. In a first aspect, the invention provides a method of reducing scar formation. In one embodiment, the scar is a glial scar. The method comprises inhibiting one or more of the following: expression of a primary proteoglycan, expression and/or activity of a GAG chain initiation enzyme, expression and/or activity of a GAG chain elongation enzyme, expression and/or activity of a GAG chain sulfation enzyme.

In one embodiment, the primary proteoglycan is selected from the group consisting of neurocan, NG2, and phosphacan. In another embodiment, the chain initiation enzyme is xylosytransferase. In another embodiment, the chain elongation enzyme is selected from the group consisting of N-acetylgalactosaminyl transferase, glucuronosyltransferase, glucosaminyltransferase, galactosaminyltransferase, N-sulfotransferase, 6-sulfotransferase, 3-sulfotransferase, 1,4-glucosaminyltransferase, 1,4-galactosaminyltransferase, N-acetylglucosamine, and glucuronic acid.

In one embodiment, the method comprises administering an agent. For example, the agent can be selected from the group consisting of antisense oligonucleotides that bind a nucleic acid sequence encoding a proteoglycan; ribozymes, DNA enzymes, RNAi constructs, and small molecules. In one embodiment, the agent is a DNA enzyme.

In a second aspect, the invention provides a method of promoting neuronal regeneration. The method comprises inhibiting one or more of the following: expression of a primary proteoglycan, expression and/or activity of a GAG chain initiation enzyme, expression and/or activity of a GAG chain elongation enzyme, expression and/or activity of a GAG chain sulfation enzyme. In one embodiment, the method comprises promoting neuronal regeneration in the presence of a glial scar.

In one embodiment, the primary proteoglycan is selected from the group consisting of neurocan, NG2, and phosphacan. In another embodiment, the chain initiation enzyme is xylosytransferase. In another embodiment, the chain elongation enzyme is selected from the group consisting of N-acetylgalactosaminyl transferase, glucuronosyltransferase, glucosaminyltransferase, galactosaminyltransferase, N-sulfotransferase, 6-sulfotransferase, 3-sulfotransferase, 1,4-glucosaminyltransferase, 1,4-galactosaminyltransferase, N-acetylglucosamine, and glucuronic acid.

In one embodiment, the method comprises administering an agent. For example, the agent can be selected from the group consisting of antisense oligunucleotides that bind a nucleic acid sequence encoding a proteoglycan; ribozymes, DNA enzymes, RNAi constructs, and small molecules. In one embodiment, the agent is a DNA enzyme.

In a third aspect, the invention provides a method of promoting neuronal regeneration comprising administering to a subject in need thereof an effective amount of an agent. An effective amount of an agent inhibits one or more of the following: expression of a primary proteoglycan, expression and/or activity of a GAG chain initiation enzyme, expression and/or activity of a GAG chain elongation enzyme, expression and/or activity of a GAG chain sulfation enzyme. In one embodiment, the method comprises promoting neuronal regeneration in the presence of a glial scar.

In one embodiment, the primary proteoglycan is selected from the group consisting of neurocan, NG2, and phosphacan. In another embodiment, the chain initiation enzyme is xylosytransferase. In another embodiment, the chain elongation enzyme is selected from the group consisting of N-acetylgalactosaminyl transferase, glucuronosyltransferase, glucosaminyltransferase, galactosaminyltransferase, N-sulfotransferase, 6-sulfotransferase, 3-sulfotransferase, 1,4-glucosaminyltransferase, 1,4-galactosaminyltransferase, N-acetylglucosamine, and glucuronic acid.

In one embodiment, the method comprises administering an agent. For example, the agent can be selected from the group consisting of antisense oligonucleotides that bind a nucleic acid sequence encoding a proteoglycan; ribozymes, DNA enzymes, RNAi constructs, and small molecules. In one embodiment, the agent is a DNA enzyme.

In another embodiment, the method further comprises administering a growth factor or neurotrophic factor. In one embodiment the growth factor or neurotrophic factor is selected from the group consisting of nerve growth factor, brain-derived growth factor, neurotrophin 3, neurotrophin 4, neurotrophin 5, glial derived neurotrophic factor, basic fibroblast growth factor, and ciliary neurotrophic factor.

In another embodiment, the method further comprises administering a proteoglycan specific enzyme.

In a fourth aspect, the invention provides a method of promoting inter-mixing of Schwann cells and astrocytes. The method comprises inhibiting one or more of the following: expression of a primary proteoglycan, expression and/or activity of a GAG chain initiation enzyme, expression and/or activity of a GAG chain elongation enzyme, expression and/or activity of a GAG chain sulfation enzyme. In one embodiment, the method comprises promoting inter-mixing of Schwann cells and astrocytes grown in culture.

In one embodiment, the primary proteoglycan is selected from the group consisting of neurocan, NG2, and phosphacan. In another embodiment, the chain initiation enzyme is xylosytransferase. In another embodiment, the chain elongation enzyme is selected from the group consisting of N-acetylgalactosaminyl transferase, glucuronosyltransferase, glucosaminyltransferase, galactosaminyltransferase, N-sulfotransferase, 6-sulfotransferase, 3-sulfotransferase, 1,4-glucosaminyltransferase, 1,4-galactosaminyltransferase, N-acetylglucosamine, and glucuronic acid.

In one embodiment, the method comprises administering an agent. For example, the agent can be selected from the group consisting of antisense oligonucleotides that bind a nucleic acid sequence encoding a proteoglycan; ribozymes, DNA enzymes, RNAi constructs, and small molecules. In one embodiment, the agent is a DNA enzyme.

In another embodiment, the method further comprises administering a growth factor or neurotrophic factor. In one embodiment the growth factor or neurotrophic factor is selected from the group consisting of nerve growth factor, brain-derived growth factor, neurotrophin 3, neurotrophin 4, neurotrophin 5, glial derived neurotrophic factor, basic fibroblast growth factor, and ciliary neurotrophic factor.

In another embodiment, the method further comprises administering a proteoglycan specific enzyme.

In a fifth aspect, the invention provides DNA enzymes that bind to and inhibit the expression and/or activitiy of a xylotransferase. In one embodiment, the DNA enzyme specifically inhibits expression and/or activity of XT-I. In another embodiment, the DNA enzyme specifically inhibits expression and/or activity of XT-II. In yet another embodiment, the DNA enzyme inhibits expression and/or activity of XT-I and XT-II.

In another embodiment, the DNA enzyme comprises a sequence set forth in SEQ ID NO: 33 or SEQ ID NO: 39.

In another embodiment, the DNA enzyme is modified.

In any of the foregoing, the invention contemplates compositions and pharmaceutical compositions comprising DNA enzymes that inhibit the expression and/or activity of a xylotransferase.

In a sixth aspect, the invention provides antisense oligonucleotides that bind to and inhibit the expression and/or activitiy of a xylotransferase. In one embodiment, the antisense oligonucleotide specifically inhibits expression and/or activity of XT-I. In another embodiment, the antisense oligonucleotide specifically inhibits expression and/or activity of XT-II. In yet another embodiment, the antisense oligonucleotide inhibits expression and/or activity of XT-I and XT-II.

In another embodiment, the antisense oligonucleotide comprises a sequence set forth in SEQ ID NO: 37 or SEQ ID NO: 38.

In another embodiment, the antisense oligonucleotide is modified.

In any of the foregoing, the invention contemplates compositions and pharmaceutical compositions comprising antisense oligonucleotides that inhibit the expression and/or activity of a xylotransferase.

In a seventh aspect, the invention provides RNAi constructs that bind to and inhibit the expression and/or activitiy of a xylotransferase. In one embodiment, the RNAi construct specifically inhibits expression and/or activity of XT-I. In another embodiment, the RNAi construct specifically inhibits expression and/or activity of XT-II. In yet another embodiment, the RNAi construct inhibits expression and/or activity of XT-I and XT-II.

In another embodiment, the RNAi construct is modified.

In any of the foregoing, the invention contemplates compositions and pharmaceutical compositions comprising RNAi constructs that inhibit the expression and/or activity of a xylotransferase.

In an eighth aspect, the invention provides methods of screening for additional agents. Agents may be screened and identified in any of a number of cell free or cell based assays. Exemplary agents have one or more of the following functions: inhibit the expression of a primary proteoglycan, inhibit the expression and/or activity of a GAG chain initiation enzyme, inhibit the expression and/or activity of a GAG chain elongation enzyme, or inhibit the expression and/or activity of a GAG chain sulfation enzyme. In one embodiment, the exemplary agent is further capable of reducing scar formation, promoting neuronal regeneration and/or promoting inter-mixing of Schwann cells and astrocytes.

In one embodiment, the method of screening comprises a method of identifying and/or characterizing an agent that has one or more of the following functions: capable of inhibiting the expression of a primary proteoglycan, capable of inhibiting the expression and/or activity of a GAG chain initiation enzyme, capable of inhibiting the expression and/or activity of a GAG chain elongation enzyme, or capable of inhibiting the expression and/or activity of a GAG chain sulfation enzyme. In another embodiment, the method of screening comprises a method of identifying and/or characterizing an agent that has one or more of the following functions: capable of reducing scar formation, capable of promoting neuronal regeneration, or capable of promoting inter-mixing of Schwann cells and astrocytes.

In another embodiment, the method of identifying and/or characterizing further comprises formulating a pharmaceutical composition/preparation comprising a candidate agent and a pharmaceutically acceptable carrier or excipient.

In still another embodiment, the pharmaceutical preparation is further tested, marketed, and sold for use in human or non-human patients. The sale to patients may also include a method for billing patients and/or their insurance provider.

In a ninth aspect, the invention provides compositions comprising agents identified by the screening methods of the present invention. In one embodiment, the compositions are pharmaceutical preparations comprising the agents and a pharmaceutically acceptable carrier or excipient.

In a tenth aspect, the invention provides a use of an agent in the manufacture of a medicament to inhibit the expression and/or activity of a xylotransferase. In one embodiment, the agent is selected from a DNA enzyme, an antisense oligonucleotide, an RNAi construct, a ribozyme, an antibody, and a morpholino construct. In another embodiment, the xylotransferase is XT-I. In yet another embodiment, the xylotransferase is XT-II.

In an eleventh aspect, the invention provides a use of an agent in the manufacture of a medicament to decrease GAG content. In one embodiment, the agent is selected from a DNA enzyme, an antisense oligonucleotide, an RNAi construct, a ribozyme, an antibody, and a morpholino construct. In another embodiment, the xylotransferase is XT-I. In yet another embodiment, the xylotransferase is XT-II.

The practice of the present invention will employ, unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

Other features and advantages of the invention will be apparent from the following detailed description, and from the claims.

### BRIEF DESCRIPTION OF THE DRAWINGS

FIG. 1 shows a schematic representation of a 10-23 DNA ribozyme. The target sequence is shown at the top, and the 10-23 enzyme is shown below. An arrow indicates the site in the target sequence where cleavage occurs. R represents either A or G, and Y represents either U or C. Note that R in the target sequence, shown in bold, is unpaired.

FIG. 2 shows a schematic representation of a 8-17 DNA enzyme. The target sequence is shown at the top, and the 10-23 enzyme is shown below. An arrow indicates the site in the target sequence where cleavage occurs. Note that A in the target sequence, shown in bold, is unpaired.

FIG. 3 provides a linear representation of an XT-I DNA enzyme.

FIG. 4 shows that treatment with XT-I DNA enzyme promoted inter-mixing of Schwann cells and astrocytes grown in co-culture in a confrontation assay.

FIG. 5 shows that treatment with XT-I DNA enzyme decreased GAG content in co-cultures of Schwann cells and astrocytes.

FIG. 6 summarizes observations of the migration of DRG cells plated on either Schwann cells or astrocytes grown in co-culture. DRG movement was influenced by the identity of the cell type on which it was initially plated.

FIG. 7 summarizes the nucleic acid and amino acid sequences provided in the sequence listing.

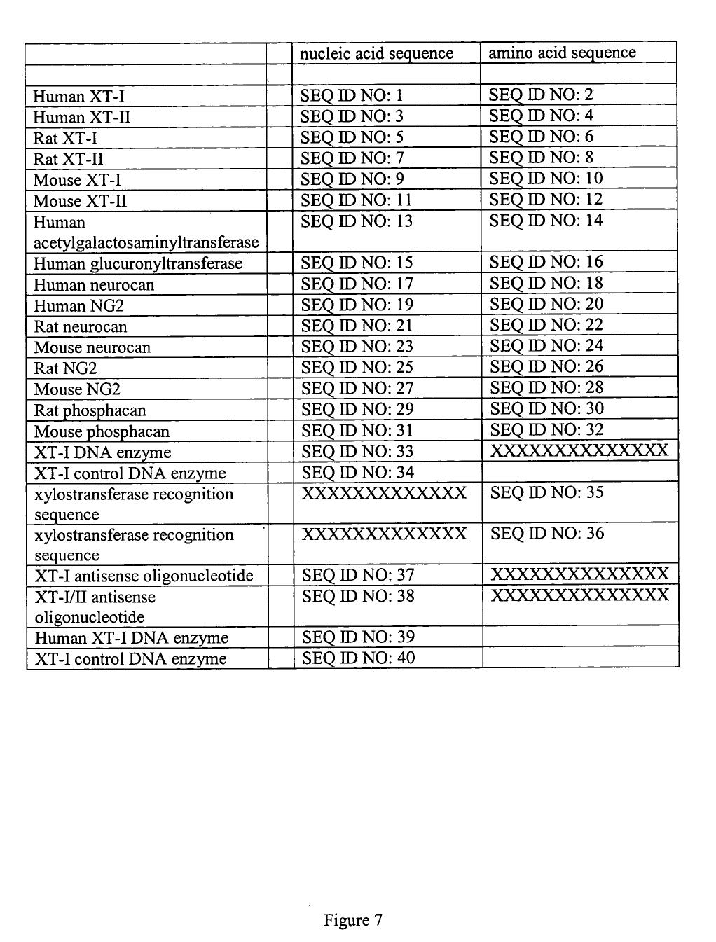

### DETAILED DESCRIPTION OF THE INVENTION

(i) Overview

The human body has tremendous regenerative capacity. However, despite the generally robust healing prowess of our bodies, neuronal tissue appears to have relatively poor regenerative capacity. In the central nervous system, cell damage frequently results in the formation of a glial scar, and this glial scar provides a physical and/or molecular barrier to regeneration. The inhibition of regeneration following injury and/or diseases of the central nervous system hampers recovery from a wide range of conditions including physical injuries to the brain or spinal cord, infections, brain or spinal cord surgery, stroke, Parkinson's disease, Huntington's disease, ALS, Alzheimer's disease, multiple sclerosis, detached retina, and macular degeneration. The ability to promote neuronal regeneration in the central nervous system would provide improved treatment options for patients afflicted with any of these conditions.

Neuronal cells of the peripheral nervous system show some limited capacity to regenerate following injury or disease. However, this regeneration occurs very slowly, and does not typically result in the restoration of total pre-injury or pre-disease function. Thus, the ability to increase the rate or extent of neuronal regeneration in the peripheral nervous system following injury or disease would greatly improve the recovery of patients afflicted with any of a number of injuries or diseases of the peripheral nervous system. Such conditions include physical injuries to cells of the peripheral nervous system, infections, neuropathy, and diabetic neuropathy.

Glial scars form in response to neuronal injury and cell damage. These glial scars comprise a combination of astrocytes, oligodendrocytes, and extracellular matrix proteins. An important component of the glial scar are proteoglycans, and many of the proteoglycans that comprise the glial scar are modified with glycosaminoglycan moieties (GAG). Proteoglycans, GAG modified proteoglycans, and GAG moieties themselves, are responsible, at least in part, for the inhibitory influence of glial scars on neuronal regeneration following injury or the cellular damage which results from disease. Given the role of proteoglycans, GAG modified proteoglycans, and GAG moieties in glial scar formation and the inhibition of neuronal regeneration, the present invention provides methods and compositions for reducing glial scar formation, methods and compositions for reducing the expression of GAG modified proteoglycans in a glial scar, and methods and compositions for reducing GAG content in a glial scar.

Proteoglycans are a family of macromolecules modified to include one or more polysaccharide (sugar) moieties. GAG containing proteoglycans are a subclass of proteoglycans characterized by the presence of one or more glycosaminoglycan chains covalently linked to the core protein via N- or O-linkages. The glycosaminoglycan chains consist of repeating disaccharides. In some instances the glycosaminoglycan chains are sulfated. The present invention provides methods and compositions for reducing glial scar formation and/or reducing the presence of GAG modification of proteoglycans in glial scar tissue by any one of several mechanisms including: inhibiting the expression of the primary proteoglycans that comprise glial scar tissue, inhibiting the expression and/or activity of an enzyme which catalyzes the initiation of GAG addition to the core protein of proteoglycans, or inhibiting the expression and/or activity of an enzyme which catalyzes the elongation of GAG chains.

There are six distinct classes of glycosaminoglycans that are distinguished based on composition, sulfation, epimerization, and acetylation: chondroitin sulfate, dermatan sulfate, heparin, heparin sulfate, hyaluronate, and keratin sulfate. In the case of chondroitin sulfate, dermatan sulfate, heparin, and heparin sulfate, the glycosaminoglycan chain is linked to a serine residue in the core protein via a xylose-galactose-galactose trisaccharide. Among these four classes of glycosaminoglycans, the sugar sequence linking the protein backbone and the GAG chain is the same, as is the biosynthetic mechanism of chain initiation. Briefly, chain initiation begins with addition of xylose from UDP-xylose to a single serine hydroxyl embedded in the specific peptide sequence QEEEEGSGGGQ (SEQ ID NO: 35). Chain initiation is catalyzed by the activity of the chain initiating xylosyltransferase (UDP-D-Xylosyltransferase). Following chain initiation, elongation of the GAG chain is catalyzed by several glycosyltransferases. These glycosyltransferases show specificity for the linkage formed, sugar moiety added, and acceptor sequence recognized, and thus the specific enzymes that catalyze each step in chain elongation varies among the classes of glycosaminoglycans.

Chondroitin sulfate consists of a series of 30-50 repeating disaccharides of N-acetylgalactosamine and glucuronic acid in a beta 1,3 linkage. The disaccharides may be optionally sulfated on the 4 or 6 position of the galactosamine residue. As outlined in detail above, chondroitin sulfate is among the four classes of glycosaminoglycans which share a common mechanism of chain initiation catalyzed by a xylosyltransferase, however each of these four classes of glycosaminoglycans differ with respect to the mechanism of chain elongation. In the case of chondroitin sulfate, chain elongation is catalyzed by several enzymes including N-acetylgalactosaminyltransferase and glucuronosyltransferase. The nucleic acid and amino acid sequence of a human N-acetylgalactosaminyltransferase is provided in SEQ ID NO: 13 and SEQ ID NO: 14, respectively. The nucleic acid and amino acid sequence of a human glucuronosyltransferase is provided in SEQ ID NO: 15 and SEQ ID NO: 16, respectively.

Methods to inhibit chondroitin sulfate modification of proteoglycans, as for example in a glial scar, can be based on, for example, inhibition of xylosyltransferase, inhibition of N-acetylgalactosaminyltransferase, or inhibition of glucuronosyltransferase. Since xylosyltransferase catalyzes chain initiation of the four classes of GAG discussed in detail above, inhibition of the activity and/or expression of xylosyltransferase would inhibit not only chondroitin sulfate modification of proteoglycans, but also other classes of GAG modification of proteoglycans. In contrast, given that the enzymes which catalyze chain elongation show greater specificity for the type of GAG modification and linkage, inhibition of the activity and/or expression of N-acetylgalactosaminyltransferase or glucuronosyltransferase would likely be more specific for inhibiting the elongation of particular GAG chains. In this case, such inhibitors may preferentially inhibit elongation of chondroitin sulfate chains. The present invention contemplates methods of inhibiting either chain initiation using inhibitors of the expression and/or activity of xylosyltransferase, methods of inhibiting chain elongation using inhibitors of N-acetylgalactosaminyltransferase and/or glucuronosyltransferase, or combinatorial methods using both inhibitors of chain initiation and inhibitors of chain elongation.

Dermatan sulfate differs from chondroitin sulfate in that glucuronic acid residues in the disaccharide chain are replaced by iduronic acid. This difference between the two GAG classes is the result of epimerization of the C5 carboxyl group and sulfation at the C2 position of glucuronic acid. Like chondroitin sulfate, dermatan sulfate is among the four classes of glycosaminoglycans which share a common mechanism of chain initiation catalyzed by a xylosyltransferase, but differ with respect to the mechanism of chain elongation. In the case of dermatan sulfate, chain elongation is catalyzed by several enzymes including N-acetylgalactosaminyltransferase. The nucleic acid and amino acid sequence of a human N-acetylgalactosaminyltransferase is provided in SEQ ID NO: 13 and SEQ ID NO: 14, respectively.

Methods to inhibit dermatan sulfate modification of proteoglycans, as for example in a glial scar, can be based on, for example, inhibition of xylosyltransferase or inhibition of N-acetylgalactosaminyltransferase. Since xylosyltransferase catalyzes chain initiation of the four classes of GAG discussed in detail above, inhibition of the activity and/or expression of xylosyltransferase would inhibit not only dermatan sulfate modification of proteoglycans, but also other classes of GAG modification of proteoglycans. In contrast, given that the enzymes which catalyze chain elongation show greater specificity for the type of GAG modification and linkage, inhibition of the activity and/or expression of N-acetylgalactosaminyltransferase would likely be more specific for inhibiting the elongation of particular GAG chains. In this case, such an inhibitor may preferentially inhibit elongation of dermatan sulfate chains. The present invention contemplates methods of inhibiting either chain initiation using inhibitors of the expression and/or activity of xylosyltransferase, methods of inhibiting chain elongation using inhibitors of N-acetylgalactosaminyltransferase, or combinatorial methods using both inhibitors of chain initiation and inhibitors of chain elongation.

Heparin and heparin sulfate are more complex than either chondroitin sulfate or dermatan sulfate. Both classes of GAGs consist of glucosamine and either glucuronic or iduonic acid in the repeating disaccharide chain. Additionally, heparin and heparin sulfate typically have a greater degree of sulfation than either chondroitin sulfate or dermatan sulfate. Both heparin and heparin sulfate may have N-acetylglucosamine acetyl groups which are N-sulfated, as well as N- or O-sulfation of the hexosamine C6 or C3 positions and O-sulfation of the uronic acid C2 positions. Typically, heparin sulfate has more N-acetyl groups and fewer N- or O-sulfate groups than heparin. Like chondroitin sulfate and dermatan sulfate, heparin and heparin sulfate are among the four classes of glycosaminoglycans which share a common mechanism of chain initiation catalyzed by a xylosyltransferase, but differ with respect to the mechanism of chain elongation. In the case of heparin and heparin sulfate, chain elongation is catalyzed by several enzymes including glucosaminyltransferase. Additionally, given the complex sulfation of heparin and heparin sulfate, the activity of several sulfotransferases, including N-sulfotransferases, 6-sulfotransferases, and 3-sulfotransferases, are important in the biosynthesis of these GAGs.

Methods to inhibit heparin and heparin sulfate modification of proteoglycans, as for example in a glial scar, can be based on, for example, inhibition of xylosyltransferase, inhibition of glucosaminyltransferase, or inhibition of one or more sulfotransferases. Since xylosyltransferase catalyzes chain initiation of the four classes of GAG discussed in detail above, inhibition of the activity and/or expression of xylosyltransferase would inhibit not only herparin and heparin sulfate modification of proteoglycans, but also other classes of GAG modification of proteoglycans. In contrast, given that the enzymes which catalyze chain elongation show greater specificity for the type of GAG modification and linkage, inhibition of the activity and/or expression of glucosaminyltransferase would likely be more specific for inhibiting the elongation of particular GAG chains. In this case, such inhibitors may preferentially inhibit herparin and heparin sulfate chains. The present invention contemplates methods of inhibiting either chain initiation using inhibitors of the expression and/or activity of xylosyltransferase, methods of inhibiting chain elongation using inhibitors of glucosaminyltransferase, methods of inhibiting sulfation using one or more sulfotransferase, and combinatorial methods using inhibtitors that act at two or more steps in heparin or heparin sulfate biosynthesis.

Keratan sulfate and hyaluronate are two class of glycosaminoglycans that differ from the other four classes in that they do not share a common mechanism of chain initiation catalyzed by xylotransferase. Keratan sulfate GAG chains do not contain uronic acid, and the repeating disaccharide unit consists of galactose and N-acetylglucosamine in beta 1,4 linkage. The covalent linkage of the GAG chain to the core protein is found in two varieties: N-linked through N-acetylglucosamine to asparagines, and O-linked through N-acetylgalactosamine to either serine or threonine. The keratin sulfate disaccharide chain also contains mannose, fucose, and/or sialic acid. In addition, keratin sulfates may be sulfated, and the degree of sulfation varies. Sulfation is typically on either galactose or hexosamine in the C6 position.

Unlike the four classes of GAGs described above, keratan sulfate does not share a common mechanism of chain initation. Nevertheless, like all GAGs keratan sulfate modification of proteoglycans (i.e., keratan sulfate biosynthesis) requires chain initiation, chain elongation, and optionally chain sulfation. Accordingly, methods to inhibit keratan sulfate modification of proteoglycans, as for example in a glial scar, can be based on, for example, inhibition of chain initiation by inhibiting the enzyme which catalyzes keratan sulfate chain initiation, inhibition of one or more of the 1,4-glucosaminyltransferases and 1,4-galactosaminyltransferases which catalyze chain elongation, or inhibition of one or more sulfotransferases.

Given that keratan sulfate chain initiation is not catalyzed by the same xylosyltransferases which catalyze chain initiation of many other classes of GAGs, inhibition of an enzyme which catalyzes keratan sulfate chain initiation would specifically inhibit keratan sulfate biosynthesis. Additionally, chain elongation during keratan sulfate biosynthesis occurs predominantly via a beta 1,4 linkage rather than via a beta 1,3 linkage, as observed for many other classes of GAGs. Thus, inhibition of the 1,4-glucosaminyltransferases and/or 1,4-galactosaminyltransferases which catalyze chain elongation during keratan sulfate biosynthesis would specifically inhibit keratan sulfate biosynthesis.

The present invention contemplates methods of inhibiting either chain initiation using inhibitors of the expression and/or activity of a keratan sulfate chain initiation enzyme, methods of inhibiting chain elongation using inhibitors of one or more 1,4-glucosaminyltransferase or 1,4-galactosaminyltransferase, methods of inhibiting sulfation using one or more sulfotransferases, and combinatorial methods using inhibtitors that act at two or more steps in keratan sulfate biosynthesis.

The sixth class of GAGs is hyaluronate. Hyaluronate differs substantially from the other five classes of GAGs in two principle ways. Firstly, hyaluronate is not sulfated, and thus inhibitation of sulfation does not provide a mechanism to inhibit hyaluronate biosynthesis. Secondly, hyaluronate is not found covalently linked to a protein core. However, despite these differences hyaluronate is structurally similar to other classes of GAGs and consists of repeating disaccharides of N-acetylglucosamine and glucuronic acid.

Given that hyaluronate is not covalently appended to a protein core, its biosynthesis does not require a specific enzyme which catalyzes chain initiation, and thus inhibition of chain initiation is not a mechanism by which to decrease hyaluronate in a glial scar. However, the presence of hyaluronate, for example in a glial scar, requires chain elongation which occurs via a mechanism similar to that used for chain elongation during biosynthesis of other GAGs. Accordingly, inhibition of one or more of the glucosaminyltransferases or glucuronosyltransferases which catalyze chain elongation during hyaluronate biosynthesis would specifically inhibit hyaluronate biosynthesis.

The present invention contemplates methods of inhibiting chain elongation using inhibitors of the expression and/or activity of a hyaluronate chain elongation enzyme including one or more glucosaminyltransferases or glucuronosyltransferases.

Previous studies have demonstrated that GAGs, either alone or appended to proteoglycans, inhibit neuronal regeneration following injury or disease. As outlined in detail above, the present invention contemplates promoting neuronal regeneration by decreasing GAG content in a glial scar. By inhibiting biosynthesis of one or more classes of GAGs and thereby decreasing GAG content in a glial scar, the present invention provides methods of decreasing the inhibitory influences of a glial scar on neuronal regeneration.

In addition to methods and compositions directed to GAGs, the present invention further contemplates that the inhibitory influence of a glial scar on neuronal regeneration can also be decreased by decreasing the expression of one or more of the proteoglycans which are highly expressed in a glial scar. Without being bound by theory, the inhibitory influences of a glial scar on neuronal regeneration are mediated by some combination of GAG modified proteoglycans and GAGs. The inhibitory influence of the glial scar on neuronal regeneration can be decreased by decreasing GAG biosynthesis, by decreasing expression of the proteoglycans which are modified by GAGs and comprise the glial scar, or by a combination of the two approaches.

By way of example, glial scars often contain one or more of the following proteoglycans: neurocan, NG2, and phosphacan. These proteoglycans are modified with GAGs. In fact, neurocan is sometimes referred to as chondroitin sulfate proteoglycan-3 (CSPG3) and NG2 is sometimes referred to as chondroitin sulfate proteoglycan-4 (CSPG4).

The nucleic acid and amino acid sequences, respectively, corresponding to human (SEQ ID NO: 17 and SEQ ID NO: 18), rat (SEQ ID NO: 21 and SEQ ID NO: 22) and mouse (SEQ ID NO: 23 and SEQ ID NO: 24) neurocan are provided herein. The present invention contemplates a variety of methods and compositions that inhibit the expression of neurocan. By decreasing the expression of neurocan, for example, the expression of neurocan at the site of neuronal injury and/or in a glial scar, the present invention further contemplates that inhibition of neurocan expression will promote neuronal regeneration.

The nucleic acid and amino acid sequences, respectively, corresponding to human (SEQ ID NO: 19 and SEQ ID NO: 20), rat (SEQ ID NO: 25 and SEQ ID NO: 26) and mouse (SEQ ID NO: 27 and SEQ ID NO: 28) NG2 are provided herein. The present invention contemplates a variety of methods and compositions that inhibit the expression of NG2. By decreasing the expression of NG2, for example, the expression of NG2 at the site of neuronal injury and/or in a glial scar, the present invention further contemplates that inhibition of NG2 expression will promote neuronal regeneration.

The nucleic acid and amino acid sequences, respectively, corresponding to rat (SEQ ID NO: 29 and SEQ ID NO: 30) and mouse (SEQ ID NO: 31 and SEQ ID NO: 32) phosphacan are provided herein. The present invention contemplates a variety of methods and compositions that inhibit the expression of phosphacan. By decreasing the expression of phosphacan, for example, the expression of phosphacan at the site of neuronal injury and/or in a glial scar, the present invention further contemplates that inhibition of phosphacan expression will promote neuronal regeneration.

Given that injured and diseased neuronal tissue typically exhibits low regenerative capacity, there exists a need for improved methods of promoting neuronal regeneration in cells of the central and peripheral nervous system. The present invention provides methods and compositions for decreasing glial scar formation, methods and compositions for decreasing GAG modification of proteoglycans that comprise glial scars, and methods and compositions for promoting neuronal regeneration. The methods and compositions provided herein may be used following injury or diseases of neuronal cells of either the central nervous system or the peripheral nervous system.

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, “glial scar” refers to the reactive gliosis which occurs following injury or degeneration of a neuron in the nervous system. The scar is composed of both glial cells and many extracellular matrix molecules including proteoglycans and glycoaminoglycans.

The terms “glial scar” and “proteoglycan rich glial scar” and “glycosaminoglycan rich glial scar” are used interchangeably throughout this application. Gliosis or glial scarring following tissue injury inhibits regeneration of cells of the nervous system following injury and other cell damage such as cell damage caused by degenerative disease. Without being bound by theory, glial scarring is thought to provide a physical and/or molecular barrier to regeneration. Exemplary glial scars comprise GAG modified proteoglycans such as chondroitin sulfate proteoglycans, heparin sulfate proteoglycans, heparin, dermatan sulfate proteoglycans, and keratin sulfate proteoglycans, as well as GAGs such as hyaluronate.

As used herein, “proteoglycan” refers to proteins modified to include one or more polysaccharide (sugar) moieties. Glycosaminoglycans are the polysaccharide chains of proteoglycans, and contain repeating units of disaccharides that consist of an amino sugar derivative. A negatively charged carboxylate or sulfate group is sometimes found in at least one of the sugar units of the disaccharide. The term proteoglycan does not refer to a single protein or to a set of proteins that share a high degree of amino acid identity. Rather, the term is used to refer to a heterogeneous “family” of proteins which may share little amino acid similarity with respect to the core protein. Proteoglycans are found in abundance in extracellular matrix and in healthy tissues including connective tissue. Additionally, a variety of studies have demonstrated that glial scars are rich in a number of extracellular matrix proteins including proteoglycans, GAG-modified proteoglycans, and GAGs (Stichel and Muller (1998) Cell Tissue Research 294: 1-9). Extracellular matrix proteins and proteoglycans found in glial scars include, without limitation, collagen IV, laminin, fibronectin, chondroitin sulfate proteoglycans, dermatan sulfate proteoglycans, heparin sulfate proteoglycans, heparin, keratin sulfate proteoglycans, hyaluronate and thrombospondin.

As used herein, “chondroitin sulfate proteoglycans (CSPGs)” and “chondroitin sulfate glycosaminoglycans (CS-GAGs)” refer to a heterogenous family of proteoglycans, as well as a heterogeneous family of glycosaminoglycan modifications. One of skill in the art will recognize that such proteoglycans are loosely classified based on modification, and the core proteins vary largely in terms of linear amino acid sequence. Additionally the extent of glycosylation can vary greatly among these proteoglycans. Examples of chondroitin sulfate proteoglycans are well known in the art, and the amino acid sequence corresponding to the core protein of many of these CSPGs has been determined in different mammalian species. Exemplary CSPGs include versican/CSPG2 [Gen Bank Accession Nos. NM_{—}004385 (human), NM_{—}019389 (mouse)], aggregan/CSPG1 [GenBank Accession Nos. NM_{—}001135 (human), NM_{—}013227 (human), NM_{—}022190 (rat)], bamacan/CSPG6 [GenBank Accession Nos. NM_{—}005445 (human), NM_{—}031583 (rat), NM_{—}007790 (mouse)], CSPG4 [GenBank Accession Nos. NM_{—}001897 (human), NM_{—}139001 (mouse)], brevican [GenBank Accession Nos. NM_{—}021948 (human), NM_{—}012916 (rat)], neuroglycanC/CSPG5 [GenBank Accession Nos. NM_{—}006574 (human), NM_{—}019284 (rat), NM_{—}013884 (mouse)], neurocan/CSPG3 [GenBank Accession Nos. NM_{—}004386 (human), NM_{—}031653 (rat), NM_{—}007789 (mouse)], leprecan [GenBank Accession Nos. AF087433 (rat)], and phosphacan [GenBank Accession Nos. U04998 (rat)]. Furthermore, chondroitin sulfate proteoglycans can potentially be composed of at least five different types of chondroitin sulfate-glycosaminoglycan (CS-GAG) chains (known as CS-GAGs A, B, C, D, and E), that differ from each other by the number and type of individual disaccharide moieties.

As used herein, “dermatan sulfate proteoglycans (DSPGs)” refer to a heterogenous family of proteoglycans, as well as a heterogeneous family of glycosaminoglycan modifications. One of skill in the art will recognize that such proteoglycans are loosely classified based on modification, and the core proteins vary largely in terms of linear amino acid sequence. Additionally the extent of glycosylation can vary greatly among these proteoglycans. Examples of dermatan sulfate proteoglycans are well known in the art, and the amino acid sequence corresponding to the core protein of many of these DSPGs has been determined in different mammalian species. Exemplary DSPGs include biglycan [GenBank Accession Nos. P21810 (human), P28653 (mouse)] and decorin/DSPG2 [GenBank Accession Nos. P07585 (human), Q01129 (rat), P28654 (mouse), AF038127 (horse)].

As used herein, “keratan sulfate proteoglycans (KSPGs)” refer to a heterogenous family of proteoglycans, as well as a heterogeneous family of glycosaminoglycan modifications. One of skill in the art will recognize that such proteoglycans are loosely classified based on modification, and the core proteins vary largely in terms of linear amino acid sequence. Additionally the extent of glycosylation can vary greatly among these proteoglycans. Examples of keratan sulfate proteoglycans are well known in the art, and the amino acid sequence corresponding to the core protein of many of these KSPGs has been determined in different mammalian species. Exemplary KSPGs include lumican [GenBank Accession Nos. P51884 (human), P51885 (mouse), P51886 (rat)]; osteomodulin [GenBank Accession Nos. Q99983 (human), 035103 (mouse), Q9Z1S7 (rat)]; fibromodulin [GenBank Accession Nos. Q06828 (human), P50608 (mouse), P50609 (rat)]; and keratocan [GenBank Accession Nos. 060938 (human) and 035367 (mouse)].

An agent which decreases the expression and/or activity of a given protein is an “inhibitor”. In the present context, exemplary inhibitors may decrease the expression and/or activity of one or more enzymes that catalyze the initiation, elongation, or sulfation of one or more GAGs. Such inhibitors may include antagonistic variants which bind to and inhibit (e.g., competitively or non-competitively inhibit) the activity of a particular enzyme which catalyzes GAG chain initiation, GAG chain elongation, or GAG chain sulfation. Additional inhibitors may include antibodies which block the activity of a particular enzyme, antisense oligonucleotides which bind to the nucleic acid sequence encoding a particular enzyme, ribozymes which catalyze the degradation of the nucleic acid sequence encoding a particular enzyme, DNA enzymes which catalyze the degradation of the nucleic acid sequence encoding a particular enzyme, RNAi constructs which bind to the nucleic acid encoding a particular enzyme, and small organic molecules which bind to and inhibit the expression and or activity of a particular enzyme. In the present context, other exemplary inhibitors may decrease the expression of one or more proteoglycans that are expressed in a glial scar. Such inhibitors may include antisense oligonucleotides which bind to the nucleic acid sequence encoding a particular proteoglycan, ribozymes which catalyze the degradation of the nucleic acid sequence encoding a particular proteoglycan, DNA enzymes which catalyze the degradation of the nucleic acid sequence encoding a particular proteoglycan, RNAi constructs which bind to the nucleic acid encoding a particular enzyme, and small organic molecules that inhibit the expression of a particular proteoglycan.

The term “agent” refers to a compound used in the methods of the present invention, as well as to a compound screened by the methods of the present invention. The term agent includes nucleic acids, peptides, proteins, peptidomimetics, small organic molecules, chemical compounds, ribozymes, RNAi constructs (including siRNA), antisense RNAs, DNA enzymes, morpholino constructs, and antibodies. Preferred agents for use in the subject methods are those which decrease glial scar formation. Further preferred agents are those which decrease GAG modification of proteoglycans. Further preferred agents are those which decrease GAG contend in a glial scar. Further preferred agents are those that decrease the expression and/or activity of any of a GAG chain initiation enzyme, a GAG chain elongation enzyme, or a GAG chain sulfation enzyme. Still further preferred agents are those which decrease expression of proteoglycans that are expressed in a glial scar. For any of these preferred examples of agents for use in the subject methods, exemplary agents may also promote inter-mixing of Schwann cells and astrocytes and/or promote neuronal regeneration.

Agents used in the methods described herein, as well as agents screened by the methods described herein can be administered and/or screened individually, or can be administered in combination with one or more other agents. Exemplary combinations include, but are not limited to, (i) one or more inhibitors of GAG chain initiation; (ii) one or more inhibitors of GAG chain elongation; (iii) one or more inhibitors of GAG chain initiation and one or more inhibitors of GAG chain elongation; (iv) one or more inhibitors of proteoglycan gene expression in a glial scar; (v) one or more inhibitors of proteoglycan gene expression in a glial scar and one or more inhibitors of GAG chain initiation; (vi) one or more inhibitors of proteoglycan gene expression in a glial scar and one or more inhibitors of GAG chain elongation; (vii) one or more inhibitors of proteoglycan gene expression in a glial scar, one or more inhibitors of GAG chain initiation, and one or more inhibitors of GAG chain elongation; (viii) one or more inhibitors of GAG chain initiation and one or more neurotrophic factors or growth factors; (ix) one or more inhibitors of GAG chain elongation and one or more neurotrophic factors or growth factors; (x) one or more inhibitors of proteoglycan gene expression in a glial scar and one or more neurotrophic factors or growth factors; (xi) one or more inhibitors of GAG chain initiation, one or more inhibitors of GAG chain elongation, and one or more neurotrophic factors or growth factors; (xii) one or more inhibitors of GAG chain initiation, one or more inhibitors of GAG chain elongation, one or more inhibitors of proteoglycan gene expression in a glial scar, and one or more neurotrophic factors or growth factors; (xiii) one or more inhibitors of GAG chain initiation and one or more proteoglycan specific enzymes; (xiv) one or more inhibitors of GAG chain elongation and one or more proteoglycan specific enzymes; (xv) one or more inhibitors of GAG chain initiation, one or more inhibitors of GAG chain elongation, and one or more proteoglycan specific enzymes; (xvi) one or more inhibitors of GAG chain initiation, one or more inhibitors of GAG chain elongation, one or more inhibitors of proteoglycan gene expression in a glial scar, and one or more proteoglycan specific enzymes; (xvii) one or more inhibitors of GAG chain sulfation; (xviii) one or more inhibitors of GAG chain sulfation and one or more inhibitors of GAG chain initiation; (xix) one or more inhibitors of GAG chain sulfation and one or more inhibitors of GAG chain elongation; (xx) one or more inhibitors of GAG chain sulfation, one or more inhibitors of GAG chain initiation, and one or more inhibitors of GAG chain elongation; (xxi) one or more inhibitor of GAG chain sulfation and one or more neurotrophic factors or growth factors; (xxii) one or more inhibitors of GAG chain sulfation and one or more inhibitors of proteoglycan gene expression in a glial scar; (xxiii) one or more inhibitors of GAG chain sulfation and one or more proteoglycan specific enzymes; (xxiv) one or more agents that promote neuronal regeneration. In any of the foregoing examples, “combinations of agents” is understood to include two or more different agents which act to inhibit the same target (e.g., two or more agents which inhibit the same biosynthetic enzyme or two or more agents which inhibit expression of the same proteoglycan), as well as combinations of agents which act to inhibit different targets (e.g., one or more agents which inhibit an enzyme involved in GAG chain initiation plus one or more agents which inhibit an enzyme involved in GAG chain elongation; one or more agents which inhibit an enzyme involved in chain elongation of a specific class of GAGs plus one or more agents which inhibit chain elongation of a specific second class of GAGs; one or more agents which inhibit the expression of a proteoglycan plus one or more agents which inhibit expression of a second proteoglycan).

The invention further contemplates the screening of libraries of agents. Such libraries may include, without limitation, cDNA libraries (either plasmid based or phage based), expression libraries, combinatorial libraries, chemical libraries, phage display libraries, variegated libraries, and biased libraries. The term “library” refers to a collection of nucleic acids, proteins, peptides, chemical compounds, small organic molecules, or antibodies. Libraries comprising each of these are well known in the art. Exemplary types of libraries include combinatorial, variegated, biased, and unbiased libraries. Libraries can provide a systematic way to screen large numbers of nucleic acids, proteins, peptides, chemical compounds, small organic molecules, or antibodies. Often, libraries are sub-divided into pools containing some fraction of the total species represented in the entire library. These pools can then be screened to identify fractions containing the desired activity. The pools can be further subdivided, and this process can be repeated until either (i) the desired activity can be correlated with a specific species contained within the library, or (ii) the desired activity is lost during further subdivision of the pool of species, and thus is the result of multiple species contained within the library. Agents may be screened and identified in any of a number of cell free or cell based assays. Exemplary agents have one or more of the following functions: inhibit the expression of a primary proteoglycan, inhibit the expression and/or activity of a GAG chain initiation enzyme, inhibit the expression and/or activity of a GAG chain elongation enzyme, or inhibit the expression and/or activity of a GAG chain sulfation enzyme. Exemplary agents may additionally possess one or more of the following functions: capable of reducing scar formation, capable of promoting neuronal regeneration, and/or capable of promoting inter-mixing of Schwann cells and astrocytes.

As used herein, “neurotrophic factor” or “growth factor” refers to any of a number of proteins known in the art that have been demonstrated to promote neuronal survival. Without being bound by theory, it is believed that neuronal survival is influenced by the presence of survival factors, and that these survival factors are present in quantities which limit the number of neurons which can survive. The remaining neurons, which appear to be otherwise normal and healthy, die. Exemplary neurotrophic factors include, without limitation, nerve growth factor (NGF), brain-derived growth factor (BDGF), neurotrophin 3 (NT-3), neurotrophin 4 (N-4), neurotrophin 5 (NT-5), glial derived neurotrophic factor (GDNF), and ciliary neurotrophic factor (CNTF). Exemplary growth factors include basic fibroblast growth factor (bFGF).

“Regeneration” of neurons and “neuronal regeneration” are used interchangeably throughout and refer to the promotion of neuronal cell growth, and/or neuronal survival following injury or cell damage. The term neuronal cell growth is meant to include extension of cellular processes including axons and dendrites. By regeneration is meant to include promotion of neuronal cell survival and/or neuronal cell growth at the site of injury (e.g., survival or growth of the injured cell itself), as well as promotion of neuronal cell survival and/or neuronal cell growth at some distance from, but in response to, the injury or cell damage.

As used herein, “neuronal cell” or “cell of the nervous system” include both neurons and glial cells.

As used herein, “CNS neuron” refers to a neuron whose cell body is located in the central nervous system. The term is also meant to encompass neurons whose cell body was originally located in the central nervous system (e.g., endogenously located in the CNS), but which have been explanted and cultured ex vivo, as well as the progeny of such cells. Examples of such neurons are motor neurons, interneurons and sensory neurons including retinal ganglion cells, dorsal root ganglion cells and neurons of the spinal cord.

As used herein, “central nervous system” refers to any of the functional regions of the brain or spinal cord. This definition is used commonly in the art and is based, at least in part, on the common embryonic origin of the structures of the brain and spinal cord from the neural tube.

The “peripheral nervous system” can be distinguished from the central nervous system, at least in part, by its differing origin during embryogenesis. Cells of the peripheral nervous system are derived from the neural crest and include neurons and glia of the sensory, sympathetic and parasympathetic systems.

As used herein, “soma” refers to the cell body of a neuron.

As used herein, “axon” and “neurite” are used interchangeably to refer to the single outgrowth which extends from a neuron and which will ultimately migrate to innervate a target tissue. The tip of the axon is referred to as the “growth cone”. Axons extend from a neuron to a target tissue, and are capable of conducting impulses. In the literature, the term “axon” is often used to refer to the outgrowth from a cell in vivo, and the term “neurite” is often used to refer to the outgrowth from a cell in vitro, however, the terms are used interchangeably herein without regard to whether the cells are found in vivo or in vitro.

As used herein, “dendrite” refers to the fine extensions from a neuron soma which pick up electrical and chemical impulses. The number of dendrites found on a given neuron vary extensively and depend on the specific neuron. Typical neurons may have multiple dendrites, but only a single axon, and it is the axon that migrates in response to cues to innervate a target tissue.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” and “nucleic acid sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

“Homology” and “identity” are used synonymously throughout and refer to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

The “non-human animals” of the invention include mammals such as rats, mice, rabbits, sheep, cats, dogs, cows, pigs, and non-human primates.

The term “isolated” as used herein with respect to nucleic acids, such as DNA or RNA, refers to molecules separated from other DNAs, or RNAs, respectively, that are present in the natural source of the macromolecule. For example, an isolated nucleic acid preferably includes no more than 10 kilobases (kb) of nucleic acid sequence which naturally immediately flanks the gene in genomic DNA, more preferably no more than Skb of such naturally occurring flanking sequences, and most preferably less than 1.5 kb of such naturally occurring flanking sequence. The term isolated as used herein also refers to a nucleic acid or peptide that is substantially free of cellular material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, “proliferating” and “proliferation” refer to cells undergoing mitosis.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agent, material, or composition comprising one or more agents as described herein which is effective for producing some desired effect in a subject; for example, an amount of the compositions described herein effective to promote neuronal regeneration, an amount effective to decrease glial scar formation, an amount effective to decrease GAG modification of proteoglycans, an amount effective to decrease GAG content in a glial scar, and/or an amount effective to decrease expression of a proteoglycan expressed in a glial scar. In a preferred embodiment, an effective amount promotes neuronal regeneration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

(iii) Exemplary Compositions

The present invention contemplates compositions and pharmaceutical compositions comprising one or more agents of the present invention. The present invention contemplates that numerous agents can be used to inhibit the expression and/or activity of an enzyme that catalyzes GAG chain initiation, GAG chain elongation, or GAG chain sulfation, as well as that numerous agents can be used to inhibit the expression of a proteoglycan that comprises a glial scar. Agents which inhibit the expression and/or activitiy of an enzyme that catalyzes GAG chain initiation, GAG chain elongation, or GAG chain sulfation, and agents that inhibit expression of a proteoglycan that comprises a glial scar can be used in the methods of the present invention. Exemplary a gents will have one or more of the following functions: (a) decrease glial scar formation, (b) decrease GAG content in a glial scar, (c) decrease proteoglycan expression in a glial scar, (d) promote neuronal regeneration, or (e) promote inter-mixing of Schwann cells and astrocytes. Further exemplary agents promote neuronal regeneration in the presence or in the absence of a glial scar.

A. Classes of Inhibitors

Numerous mechanisms exist to inhibit the expression and/or activity of a particular mRNA or protein. Without being bound by theory, the present invention contemplates any of a number of methods for inhibiting the expression and/or activity of a particular mRNA. Furthermore, the invention contemplates any of a number of methods for inhibiting the expression and/or activity of a particular protein. Still furthermore, the invention contemplates combinatorial methods comprising either (i) the use of two or more inhibitors that decrease the expression and/or activity of a particular mRNA or protein, or (ii) the use of one or more inhibitors that decrease the expression and/or activity of a particular mRNA or protein plus the use of one or more inhibitors that decrease the expression and/or activity of a second mRNA or protein.

The following are illustrative examples of methods for inhibiting the expression and/or activity of an mRNA or protein. These examples are in no way meant to be limiting, and one of skill in the art can readily select from among known methods of inhibiting expression and/or activity.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

Ribozymes molecules designed to catalytically cleave an mRNA transcripts can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

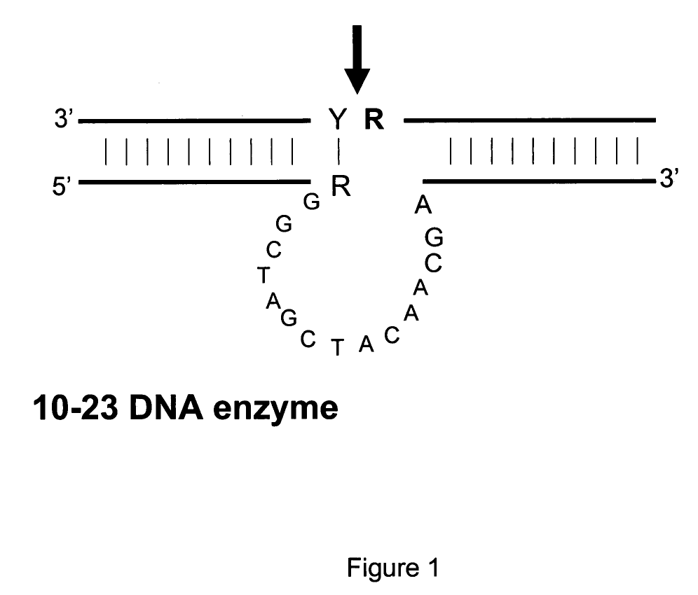

There are currently two basic types of DNA enzymes, and both of these were identified by Santoro and Joyce (see, for example, U.S. Pat. No. 6,110,462). The 10-23 DNA enzyme (shown schematically in FIG. 1) comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

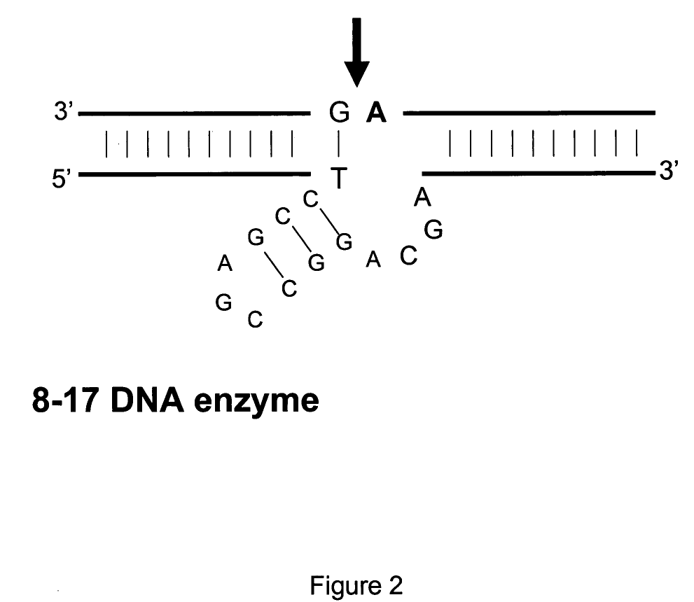

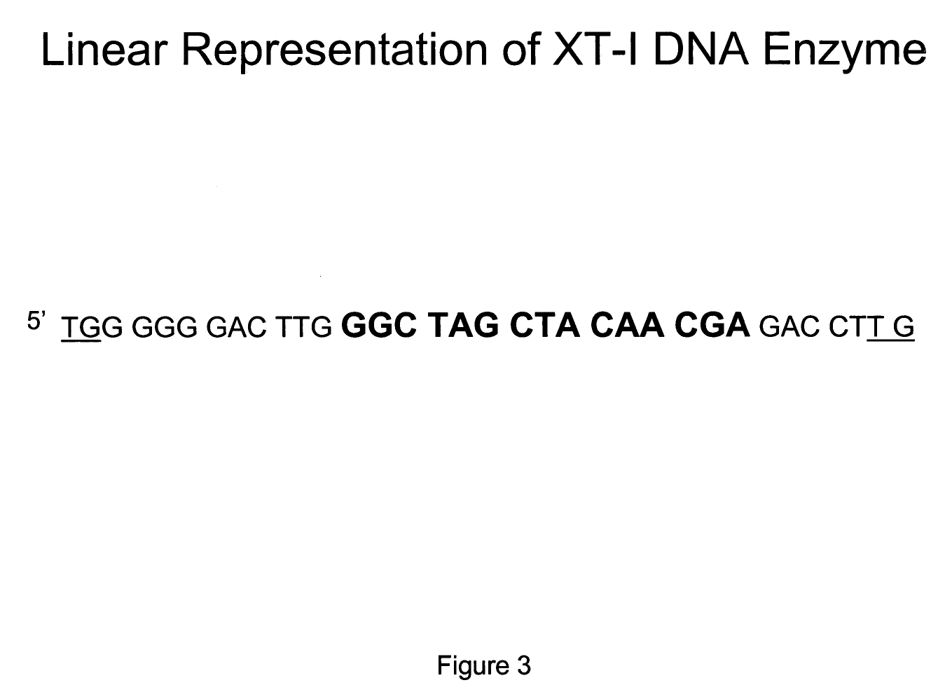

When synthesizing the DNA enzyme, the specific antisense recognition sequence that will target the enzyme to the message is divided so that it comprises the two arms of the DNA enzyme, and the DNA enzyme loop is placed between the two specific arms. Schematic representation of DNA enzymes are provided in FIGS. 1 and 2. Additionally, FIG. 3 provides the sequence of a specific DNA enzyme—an XT-I DNA enzyme.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antibodies can be used as inhibitors of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of the protein by sterically hindering the proper protein-protein interactions or occupying active sites. Alternatively the binding of the antibody to an epitope on the particular protein may alter the conformation of that protein such that it is no longer able to properly function. In the context of the present application, a preferred antibody may bind to and inhibit the function of an enzyme involved in GAG chain initiation, GAG chain elongation, or GAG chain sulfation. Such an antibody may function by binding at or near the active site of the particular enzyme such that the enzyme is unable to properly function. Alternatively, the antibody may bind to a different site on the enzyme to sterically hinder the protein-protein interactions required for enzyme function. In still another example, the antibody may bind to a different site on the enzyme and alter the conformation of the enzyme such that the enzyme is no longer able to function.

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

In the context of the present invention, antibodies can be screened and tested to identify those antibodies that can inhibit the function of a particular protein. One of skill in the art will recognize that not every antibody that is specifically immunoreactive with a particular protein will interfere with the function of that protein. However, one of skill in the art can readily test antibodies to identify those that are capable of blocking the function of a particular protein.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular protein in a biological process, or in a therapeutic context.

In addition to the use of antibodies to inhibit the function of, for example, an enzyme involved in GAG chain initiation, an enzyme involved in GAG chain elongation, or an enzyme involved in GAG chain sulfation, the present invention contemplate that antibodies raised against a particular protein can also be used to monitor the expression of that protein in vitro or in vivo (e.g., such antibodies can be used in immunohistochemical staining). In any of the foregoing, the invention contemplates that antibodies can be readily humanized to make them suitable for administration to human patients.

Variants polypeptides and peptide fragments can antagonize the function of a particular protein. Examples of such variants and fragments include dominant negative mutants of a particular protein. Antagonistic variants may function in any of a number of ways, for example, as described herein. One of skill in the art can readily make variants comprising an amino acid sequence at least 60%, 70%, 75%, 80%, 85%, 90%, 95%, 98% or 99% identical to a particular, and identify variants that antagonize the function of the wildtype protein. Further examples of antagonistic variants and antagonistic peptide fragments are described in the present application.

Small organic molecules can antagonize the function of a particular protein. By small organic molecule is meant a carbon contain molecule having a molecular weight less than 2500 amu, more preferably less than 1500 amu, and even more preferably less than 750 amu. In the context of the present invention, such small organic molecules would be able to inhibit the activity of an enzyme that catalyzes GAG chain initiation, an enzyme that catalyzes GAG chain elongation, or an enzyme that catalyzes GAG chain sulfation. In a preferred embodiment, such small organic molecules promote neuronal regeneration.

Small organic molecules can be readily identified by screening libraries of organic molecules and/or chemical compounds to identify those compounds that have a desired function. Without being bound by theory, small organic molecules may exert their inhibitory function in any of a number of ways. For example, a small organic molecules may bind to the active site of an enzyme that catalyzes GAG chain initiation, GAG chain elongation, or GAG chain sulfation, and thereby inhibit the function of that enzyme. Similarly, the small organic molecule may bind to and alter the confirmation of the enzyme, and thus inhibit the function of that enzyme. In another example, the small organic molecules may bind to another site on the enzyme and disrupt an interaction required for the functionality of the enzyme. To illustrate, an enzyme may require a protein, vitamin, metal, or other cofactor for functionality, and the small organic molecule may disrupt this interaction.

In addition to screening readily available libraries to identify small organic molecules with a particular inhibitory function, the present invention contemplates the rational design and testing of small organic molecules that can inhibit the function of a particular enzyme. For example, based on molecular modeling of the binding site of a particular enzyme, one of skill in the art can design small molecules that can occupy that binding pocket. Such small organic molecules would be candidate inhibitors of the function of the particular enzyme.

The present invention contemplates a large number of agents that function as inhibitors including nucleic acid, peptide, polypeptide, small organic molecule, antisense oligonucleotide, RNAi construct, antibody, ribozyme, and DNA enzyme based agents that function as inhibitors. Exemplary inhibitors include agents that inhibit the expression and/or activity of an enzyme involved in GAG chain initiation, an enzyme involved in GAG chain elongation, or an enzyme involved in GAG chain sulfation. Further exemplary inhibitors include agents that inhibit the expression of a proteoglycan that comprises a glial scar.

Agents that function as inhibitors and can be used in the methods of the present invention have one or more of the following functions: (i) decrease glial scar formation, (ii) decrease GAG content in a glial scar, (iii) decrease expression of one or more proteoglycans that comprise a glial scar, (iv) promote inter-mixing of Schwann cell and astrocytes, or (v) promote neuronal regeneration. Neuronal regeneration includes the promotion of neuronal growth and/or survival in any of the following scenarios: regeneration in the central and/or peripheral nervous system; in vivo and/or in vitro; following injury or in a disease state; in the presence or absence of a glial scar.

Without being bound by theory, an inhibitor for use in the methods of the present invention may function in any of a number of ways. Exemplary mechanisms include, but are not limited to (a) an antibody that binds to and inhibits the activity of an enzyme that catalyzes GAG chain initiation, (b) an antibody that binds to and inhibits the activity of an enzyme that catalyzes GAG chain elongation, (c) an antibody that binds to and inhibits the activity of an enzyme that catalyzes GAG chain sulfation, (d) a small organic molecule that binds to and inhibits the activity of an enzyme that catalyzes GAG chain initiation, (e) a small organic molecule that binds to and inhibits the activity of an enzyme that catalyzes GAG chain elongation, (f) a small organic molecule that binds to and inhibits the activity of an enzyme that catalyzes GAG chain sulfation, (g) an antagonistic polypeptide variant that inhibits the activity of an enzyme that catalyzes GAG chain initiation, (h) an antagonistic polypeptide variant that inhibits the activity of an enzyme that catalyzes GAG chain elongation, (i) an antagonistic polypeptide variant that inhibits the activity of an enzyme that catalyzes GAG chain sulfation, (j) an antagonistic peptide fragment that inhibits the activity of an enzyme that catalyzes GAG chain initiation, (k) an antagonistic peptide fragment that inhibits the activity of an enzyme that catalyzes GAG chain elongation, (l) an antagonistic peptide fragment that inhibits the activity of an enzyme that catalyzes GAG chain sulfation, (m) an antisense oligonucleotide that binds to and decreases the expression and/or activity of an enzyme that catalyze GAG chain initiation, (n) an antisense oligonucleotide that binds to and decreases the expression and/or activity of an enzyme that catalyze GAG chain elongation, (o) an antisense oligonucleotide that binds to and decreases the expression and/or activity of an enzyme that catalyze GAG chain sulfation, (p) an antisense oligonucleotide that binds to and decreases the expression of a proteoglycan that comprises a glial scar, (q) an RNAi construct that decreases the expression and/or activity of an enzyme that catalyze GAG chain initiation, (r) an RNAi construct that decreases the expression and/or activity of an enzyme that catalyze GAG chain elongation, (s) an RNAi construct that decreases the expression and/or activity of an enzyme that catalyze GAG chain sulfation, (t) an RNAi construct that decreases the expression of a proteoglycan that comprises a glial scar, (u) a ribozyme that binds to and decreases the expression and/or activity of an enzyme that catalyzes GAG chain initiation, (v) a ribozyme that binds to and decreases the expression and/or activity of an enzyme that catalyzes GAG chain elongation, (w) a ribozyme that binds to and decreases the expression and/or activity of an enzyme that catalyzes GAG chain sulfation, (x) a ribozyme that binds to and decreases the expression of a proteoglycan that comprises a glial scar, (y) a DNA enzyme that binds to and decreases the expression and/or activity of an enzyme that catalyzes GAG chain initiation, (z) a DNA enzyme that binds to and decreases the expression and/or activity of an enzyme that catalyzes GAG chain elongation, (aa) a DNA enzyme that binds to and decreases the expression and/or activity of an enzyme that catalyze GAG chain sulfation, and (bb) a DNA enzyme that decreases the expression of a proteoglycan that comprises a glial scar.

The foregoing mechanisms are by no means exhaustive. Agents for use in the subject methods decrease the expression and/or activity of an enzyme that catalyzes GAG chain initiation, an enzyme that catalyzes GAG chain elongation, and enzyme that catalyzes GAG chain sulfation, or a proteglycan that comprises a glial scar. Reduction in the expression and/or activity of one or more of the foregoing enzymes or proteoglycans can have one or more of the following biological effects: (i) decrease glial scar formation, (ii) decrease GAG content in a glial scar, (iii) decrease expression of proteoglycans that comprise a glial scar, (iv) promote inter-mixing of Schwann cells and astrocytes, or (v) promote neuronal regeneration. Without being bound by theory, a decrease in glial scar formation, GAG content in a glial scar, or proteoglycan expression decreases the inhibitory influences on neurite extension and neuronal regeneration exerted by modified proteoglycans and GAGs which comprises a glial scar. Accordingly, methods which decrease the level of glycosaminoglycan modification of proteoglycans promote neuronal regeneration. Furthermore, the invention contemplates that certain agents can promote neuronal regeneration in the absence of a glial scar.

To provide further illustrative examples of agents for use in the subject methods, the invention contemplates the following: (a) an antibody that binds to and inhibits the activity of an enzyme that catalyzes GAG chain initiation, wherein the enzyme is XT-I, and wherein the antibody is specifically immunoreactive with a polypeptide comprising an amino acid sequence provided in SEQ ID NO: 2, SEQ ID NO: 6, or SEQ ID NO: 10, (b) an antibody that binds to and inhibits the activity of an enzyme that catalyzes GAG chain initiation, wherein the enzyme is XT-II, and wherein the antibody is specifically immunoreactive with a polypeptide comprising an amino acid sequence provided in SEQ ID NO: 4 SEQ ID NO: 8, or SEQ ID NO: 12, (c) an antibody that binds to and inhibits the activity of an enzyme that catalyzes GAG chain initiation, wherein the antibody is specifically immunoreactive with both XT-I and XT-II, and wherein the antibody is specifically immunoreactive with a polypeptide comprising an amino acid sequence provided in SEQ ID NO: 2, SEQ ID NO: 4, SEQ ID NO: 6, SEQ ID NO: 8, SEQ ID NO: 10, or SEQ ID NO: 12, (d) an antibody that binds to and inhibits the activity of an enzyme that catalyzes GAG chain elongation, wherein the enzyme is acetylgalactosaminyltransferase, and wherein the antibody is specifically immunoreactive with a polypeptide comprising an amino acid sequence provided in SEQ ID NO: 14, (e) an antibody that binds to and inhibits the activity of an enzyme that catalyzes GAG chain elongation, wherein the enzyme is glucuronyltransferase, and wherein the antibody is specifically immunoreactive with a polypeptide comprising an amino acid sequence provided in SEQ ID NO: 16, (f) a small organic molecule that binds to and inhibits the activity of an enzyme that catalyzes GAG chain initiation, wherein the enzyme is XT-I, (g) a small organic molecule that binds to and inhibits the activity of an enzyme that catalyzes GAG chain initiation, wherein the enzyme is XT-II, (h) a small organic molecule that binds to and inhibits the activity of both XT-I and XT-II, (i) a small organic molecule that binds to and inhibits the activity of an enzyme that catalyzes GAG chain elongation, wherein the enzyme is acetylgalactosaminyltransferase, (j) a small organic molecule that binds to and inhibits the activity of an enzyme that catalyzes GAG chain elongation, wherein the enzyme is glucuronyltransferase, (k) a small organic molecule that binds to and inhibits the activity of an enzyme that catalyzes GAG chain sulfation, (l) an antagonistic polypeptide variant that inhibits the activity of an enzyme that catalyzes GAG chain initiation, wherein said enzyme is XT-I, and wherein said antagonistic polypeptide variant comprises an amino acid sequence at least 60%, 70%, 75%, 80%, 85%, 90%, 95%, 98%, or 99% identical to an amino acid sequence provided in SEQ ID NO: 2, SEQ ID NO: 6, or SEQ ID NO: 10, (m) an antagonistic polypeptide variant that inhibits the activity of an enzyme that catalyzes GAG chain initiation, wherein said enzyme is XT-II, and wherein said antagonistic polypeptide variant comprises an amino acid sequence at least 60%, 70%, 75%, 80%, 85%, 90%, 95%, 98%, or 99% identical to an amino acid sequence provided in SEQ ID NO: 4, SEQ ID NO: 8, or SEQ ID NO: 12, (n) an antagonistic polypeptide variant that inhibits the activity of XT-I and XT-II, wherein said antagonistic polypeptide variant comprises an amino acid sequence at least 60%, 70%, 75%, 80%, 85%, 90%, 95%, 98%, or 99% identical to an amino acid sequence provided in SEQ ID NO: 2, SEQ ID NO: 4, SEQ ID NO: 6, SEQ ID NO: 8, SEQ ID NO: 10, or SEQ ID NO: 12, (o) an antagonistic polypeptide variant that inhibits the activity of an enzyme that catalyzes GAG chain elongation, wherein said enzyme is acetylgalactosaminyltransferase, and wherein said antagonistic polypeptide variant comprises an amino acid sequence at least 60%, 70%, 75%, 80%, 85%, 90%, 95%, 98%, or 99% identical to an amino acid sequence provided in SEQ ID NO: 14, (p) an antagonistic polypeptide variant that inhibits the activity of an enzyme that catalyzes GAG chain elongation, wherein said enzyme is glucuronyltransferase, and wherein said antagonistic polypeptide variant comprises an amino acid sequence at least 60%, 70%, 75%, 80%, 85%, 90%, 95%, 98%, or 99% identical to an amino acid sequence provided in SEQ ID NO: 16, (q) an antagonistic peptide fragment that inhibits the activity of an enzyme that catalyzes GAG chain initiation, wherein the enzyme is XT-I, and wherein the peptide fragment comprises a fragment of an amino acid sequence provided in SEQ ID NO: 2, SEQ ID NO: 6, or SEQ ID NO: 10, (r) an antagonistic peptide fragment that inhibits the activity of an enzyme that catalyzes GAG chain initiation, wherein the enzyme is XT-II, and wherein the peptide fragment comprises a fragment of an amino acid sequence provided in SEQ ID NO: 4, SEQ ID NO: 8, or SEQ ID NO: 12, (s) antagonistic peptide fragment that inhibits the activity of an enzyme that catalyzes GAG chain elongation, wherein the enzyme is acetylgalactosaminyltransferase, and wherein the peptide fragment comprises a fragment of an amino acid sequence provided in SEQ ID NO: 14, (t) an antagonistic peptide fragment that inhibits the activity of an enzyme that catalyzes GAG chain elongation, wherein the enzyme is glucuronyltransferase, and wherein the peptide fragment comprises a fragment of an amino acid sequence provided in SEQ ID NO: 16, (u) an antisense oligonucleotide that binds to and decreases the expression and/or activity of an enzyme that catalyze GAG chain initiation, wherein said enzyme is XT-I, and wherein said antisense oligonucleotide hybridizes under stringent conditions to a nucleic acid sequence provided in at least one of SEQ ID NO: 1, SEQ ID NO: 5, or SEQ ID NO: 9, (v) an antisense oligonucleotide that binds to and decreases the expression and/or activity of an enzyme that catalyze GAG chain initiation, wherein said enzyme is XT-II, and wherein said antisense oligonucleotide hybridizes under stringent conditions to a nucleic acid sequence provided in at least one of SEQ ID NO: 3, SEQ ID NO: 7, or SEQ ID NO: 11, (w) an antisense oligonucleotide that binds to and decreases the expression and/or activity of XT-I and XT-II, wherein said antisense oligonucleotide hybridizes under stringent conditions to a nucleic acid sequence provided in at least one of SEQ ID NO: 1, SEQ ID NO: 3, SEQ ID NO: 5, SEQ ID NO: 7, SEQ ID NO: 9, or SEQ ID NO: 11, (x) an antisense oligonucleotide that binds to and decreases the expression and/or activity of an enzyme that catalyze GAG chain elongation, wherein said enzyme is acetylgalactosaminyltransferase, and wherein said antisense oligonucleotide hybridizes under stringent conditions to a nucleic acid sequence provided in at least one of SEQ ID NO: 13, (y) an antisense oligonucleotide that binds to and decreases the expression and/or activity of an enzyme that catalyze GAG chain elongation, wherein said enzyme is glucuronyltransferase, and wherein said antisense oligonucleotide hybridizes under stringent conditions to a nucleic acid sequence provided in at least one of SEQ ID NO: 15, (z) an antisense oligonucleotide that binds to and decreases the expression of a proteoglycan that comprises a glial scar, wherein said proteoglycan is selected from neurocan, NG2, and phosphacan, and wherein said antisense oligonucleotide hybridizes under stringent conditions to a nucleic acid sequence provided in at least one of SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31, (aa) a ribozyme that binds to and decreases the expression and/or activity of an enzyme that catalyzes GAG chain initiation, wherein said enzyme is XT-I, and wherein said ribozyme hybridizes under stringent conditions to a nucleic acid sequence provided in at least one of SEQ ID NO: 1, SEQ ID NO: 5, SEQ ID NO: 9, (bb) a ribozyme that binds to and decreases the expression and/or activity of an enzyme that catalyzes GAG chain initiation, wherein said enzyme is XT-II, and wherein said ribozyme hybridizes under stringent conditions to a nucleic acid sequence provided in at least one of SEQ ID NO: 3, SEQ ID NO: 7, SEQ ID NO: 11, (cc) a ribozyme that binds to and decreases the expression of a proteoglycan that comprises a glial scar, wherein said proteoglycan is selected from the neurocan, NG2, and phosphacan, and wherein said ribozyme hybridizes under stringent conditions to a nucleic acid sequence provided in at least one of SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31, (dd) a DNA enzyme that binds to and decreases the expression and/or activity of an enzyme that catalyzes GAG chain initiation, wherein said enzyme is XT-I, and wherein said DNA enzyme hybridizes under stringent conditions to a nucleic acid sequence provided in at least one of SEQ ID NO: 1, SEQ ID NO: 5, SEQ ID NO: 9, (ee) a DNA enzyme that binds to and decreases the expression and/or activity of an enzyme that catalyzes GAG chain elongation, wherein said enzyme is XT-II, and wherein said DNA enzyme hybridizes under stringent conditions to a nucleic acid sequence provided in at least one of SEQ ID NO: 3, SEQ ID NO: 7, SEQ ID NO: 11, (ff) a DNA enzyme that binds to and decreases the expression and/or activity of XT-I and XT-II, wherein said DNA enzyme hybridizes under stringent conditions to a nucleic acid sequence provided in at least one of SEQ ID NO: 1, SEQ ID NO: 3, SEQ ID NO: 5, SEQ ID NO: 7, SEQ ID NO: 9, or SEQ ID NO: 11, (gg) a DNA enzyme that comprises the sequence provided in SEQ ID NO: 33, (hh) an antisense oligonucleotide that comprises a sequence provided in SEQ ID NO: 33, (ii) a DNA enzyme that binds to and decreases the expression and/or activity of an protein that catalyzes GAG chain elongation, wherein said protein is acetylgalactoaminyltransferase, and wherein said DNA enzyme comprises a nucleic acid sequence that hybridizes under stringent conditions to a nucleic acid sequence provided in SEQ ID NO: 13, (jj) a DNA enzyme that binds to and decreases the expression and/or activity of an protein that catalyzes GAG chain elongation, wherein said protein is glucuronyltransferase, and wherein said DNA enzyme comprises a nucleic acid sequence that hybridizes under stringent conditions to a nucleic acid sequence provided in SEQ ID NO: 15, (kk) a DNA enzyme that binds to and decreases the expression and/or activity of an enzyme that catalyze GAG chain sulfation, (ll) a DNA enzyme that decreases the expression of a proteoglycan that comprises a glial scar, wherein said proteoglycan is selected from neurocan, NG2, and phosphacan, and wherein said DNA enzyme comprises a nucleic acid sequence that hybridizes under stringent conditions to a nucleic acid sequence provided in SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31, (mm) an antisense oligonulceotide that comprises the sequence provided in SEQ ID NO: 37 or SEQ ID NO: 38.

In any of the foregoing, the application contemplates that inhibitors may be administered alone, or may be administered in combination with one or more other agents. Similarly, in methods of screening for additional inhibitors the application contemplates that agents may be screened singly or in combination with one or more other agents. Such combinations include (i) combinations of two of more agents that each act to inhibit expression and/or activity of the same enzyme or proteoglycan, (ii) combinations of agents which each act to inhibit the expression and/or activity of different enzymes or proteoglycans, (iii) combinations of one or more agents that inhibit the expression and/or activity of a enzyme or proteoglycan with an agent that promotes neuronal survival, (iv) combinations of agents that inhibit the expression and/or activity of different enzymes or proteoglycans with an agent that promotes neuronal survival, (v) combinations of one or more agents that inhibit the expression and/or activity of a enzyme or proteoglycan with an enzyme that digests proteoglycan sugars, and (vi) combinations of agents that inhibit the expression and/or activity of different enzymes or proteoglycans with an enzyme that digests proteoglycan sugars. Furthermore, the invention contemplates that the inhibitors described herein can be administered in combination with a cellular transplant (e.g., transplantation of stem cells, fetal tissue, differentiated neurons, Schwann cells, astrocytes, etc.).

As described herein, one aspect of the invention pertains to variants of a wildtype enzyme, wherein the variants antagonize the function of the wildtype enzyme. Furthermore, one aspect of the invention pertains to fragments of a wildtype enzyme, wherein the fragments antagonize the function of the wildtype enzyme. Preferred antagonistic variants and antagonistic fragments have one or more of the following biological properties: (i) decrease glial scar formation, (ii) decrease GAG content in a glial scar, (iii) decrease expression of a proteoglycan that comprises a glial scar, (iv) promote inter-mixing of Schwann cells and astrocytes, or (v) promote neuronal regeneration. In a particularly preferred example, the antagonistic variant or antagonistic fragment promotes neuronal regeneration.

In addition to the antagonistic variants and antagonistic fragments, the invention contemplates nucleic acids comprising nucleotide sequences encoding such antagonistic variants and antagonistic fragments. The term nucleic acid as used herein is intended to include equivalents. The term equivalent is understood to include nucleotide sequences which are functionally equivalent to a particular nucleotide sequence. Equivalent nucleotide sequences will include sequences that differ by one or more nucleotide substitutions, additions or deletions, such as allelic variants, and variation due to degeneracy of the genetic code. Equivalent sequences may also include nucleotide sequences that hybridize under stringent conditions (i.e., equivalent to a bout 20-27° C. below the melting temperature (Tₘ) of the DNA duplex formed in about 1M salt) to a given nucleotide sequence. Further examples of stringent hybridization conditions include a wash step of 0.2×SSC at 65° C.

The present invention contemplates that antagonistic variants and peptide variants, for example, variants comprising an amino acid sequence at least 60%, 70%, 75%, 80%, 85%, 90%, 95%, 98%, or 99% identical to an amino acid sequence provided in SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16, can be encoded by a nucleic acid sequence. In one embodiment, the nucleic acid sequence is at least 60%, 70%, 75%, 80%, 85%, 90%, 95%, 98%, or 99% identical to a nucleic acid sequence provided in SEQ ID NO: 1, SEQ ID NO: 3, SEQ ID NO: 5, SEQ ID NO: 7, SEQ ID NO: 9, SEQ ID NO: 11, SEQ ID NO: 13, or SEQ ID NO: 15.

In another embodiment, the nucleic acid sequence hybridizes under stringent conditions, including a wash step of 0.2×SSC at 65° C., to a nucleic acid sequence provided in SEQ ID NO: 1, SEQ ID NO: 3, SEQ ID NO: 5, SEQ ID NO: 7, SEQ ID NO: 9, SEQ ID NO: 11, SEQ ID NO: 13, or SEQ ID NO: 15, or the complement thereof.

The present invention contemplates that methods of administering nucleic acids encoding antagonistic variants or peptide variants, wherein said nucleic acid has one or more of the following biological functions: (i) decreases glial scar formation, (ii) decreases GAG content, (iii) decreases expression of a proteoglycan that comprises a glial scar, (iv) promotes inter-mixing of Schwann cells and astrocytes, or (v) promotes neuronal regeneration. In a preferred embodiment, administering a nucleic acid encoding an antagonistic variant or peptide variant promotes neuronal regeneration.

Nucleic acids having a sequence that differs from nucleotide sequences which encode a particular antagonistic variant or antagonistic peptide fragment due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist.

In general, polypeptides referred to herein as antagonistic variants or antagonistic peptide fragments are defined as polypeptides which include an amino acid sequence corresponding (e.g., at least 60%, 70%, 75%, 80%, 85%, 90%, 95%, 98%, or 99% identical) to all or a portion of an amino acid sequence of a wildtype enzyme that catalyzes GAG chain inititation, GAG chain elongation, or GAG chain sulfation. Such antagonistic polypeptides have one or more of the following biological activities: (i) decrease glial scar formation, (ii) decrease GAG content in a glial scar, (iii) decrease expression of a proteoglycan that comprises a glial scar, (iv) promote inter-mixing of Schwann cells and astrocytes, or (v) promote neuronal regeneration. In another embodiment, the antagonistic variant promotes neuronal regeneration.

Exemplary antagonistic variant polypeptides include polypeptides comprising an amino acid sequence at least 60%, 70%, 75%, 80%, 85%, 90%, 95%, 98%, or 99% identical to at least one amino acid sequence provided in SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16. Such antagonistic polypeptides have one or more of the following biological activities: (i) decrease glial scar formation, (ii) decrease GAG content in a glial scar, (iii) decrease expression of a proteoglycan that comprises a glial scar, (iv) promote inter-mixing of Schwann cells and astrocytes, or (v) promote neuronal regeneration. In another embodiment, the antagonistic variant promotes neuronal regeneration.

(iv) Methods of Expressing Inhibitors

The systems and methods described herein also provide expression vectors containing a nucleic acid encoding an antagonist of an enzyme involved in GAG chain initiation or GAG chain elongation, operably linked to at least one transcriptional regulatory sequence. Such antagonists include variants of an enzyme which inhibits (either competitively or non-competitively) the activity of the wildtype enzyme. Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types.

Polypeptide antagonists, for use in the subject methods, include recombinant variants of an enzyme which catalyzes GAG chain initiation, GAG chain elongation, or GAG chain sulfation, wherein the variant antagonizes the activity of the wildtype enzyme. By way of example, polypeptide antagonists for use in the subject methods include variants of XT-I, XT-II, N-acetylgalactosaminyltransferase, or glucuronyltransferase, wherein said variant antagonizes the wildtype function of the enzyme (e.g., inhibits the catalysis of GAG chain initiation or GAG chain elongation). Exemplary antagonistic variant polypeptides comprise an amino acid sequence at least 60%, 70%, 75%, 80%, 85%, 90%, 95%, 98%, or 99% identical to the wildtype polypeptide. For example, an antagonistic variant of XT-I may comprise an amino acid sequence at least 60%, 70%, 75%, 80%, 85%, 90%, 95%, 98%, or 99% identical to the amino acid sequence provided in SEQ ID NO: 2, SEQ ID NO: 6, or SEQ ID NO: 10. An antagonistic variant of XT-II may comprise an amino acid sequence at least 60%, 70%, 75%, 80%, 85%, 90%, 95%, 98%, or 99% identical to the amino acid sequence provided in SEQ ID NO: 4, SEQ ID NO: 8, or SEQ ID NO: 12. An antagonistic variant of N-acetylgalactosaminyltransferase may comprise an amino acid sequence at least 60%, 70%, 75%, 80%, 85%, 90%, 95%, 98%, or 99% identical to the amino acid sequence provided in SEQ ID NO: 14. An antagonistic variant of glucuronyltransferase may comprise an amino acid sequence at least 60%, 70%, 75%, 80%, 85%, 90%, 95%, 98%, or 99% identical to the amino acid sequence provided in SEQ ID NO: 16. Further antagonistic variants may comprise an inhibitory fragment of the wildtype enzyme that catalyzes GAG chain initiation or GAG chain elongation. Such peptide fragments comprise at least 10, 15, 20, 25, 50, 75, 100, 150, 200, or greater than 200 amino acid residues of a wildtype enzyme.

In one example, the application provides a method of promoting neuronal regeneration, comprising administering an antagonistic variant wherein the antagonistic variant comprises an amino acid sequence at least 60%, 70%, 80%, 85%, 90%, 95%, 98%, or 99% identical to an amino acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16. In another embodiment, the application provides a method of inhibiting glial scar formation, comprising administering an antagonistic variant wherein the antagonistic variant comprises an amino acid sequence at least 60%, 70%, 80%, 85%, 90%, 95%, 98%, or 99% identical to an amino acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16. In still another embodiment, the application provides a method of decreasing GAG content in a glial scar, comprising administering an antagonistic variant wherein the antagonistic variant comprises an amino acid sequence at least 60%, 70%, 80%, 85%, 90%, 95%, 98%, or 99% identical to an amino acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16. In yet another embodiment, the application provides a method of promoting intermixing of Schwann cells and astrocytes, comprising administering an antagonistic variant wherein the antagonistic variant comprises an amino acid sequence at least 60%, 70%, 80%, 85%, 90%, 95%, 98%, or 99% identical to an amino acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16.

Furthermore, the present invention contemplates that recombinant variant polypeptides that antagonize the function of a wildtype enzyme that catalyzes GAG chain initiation, GAG chain elongation, or GAG chain sulfation are encoded by a variant nucleic acid sequence. Such variant nucleic acid sequences may be, for example, at least 60%, 70%, 75%, 80%, 85%, 90%, 95%, 98%, or 99% identical to a nucleic acid sequence encoding a wildtype enzyme. For example, the invention contemplates that exemplary antagonistic variants are encoded by nucleic acid sequences at least 60%, 70%, 75%, 80%, 85%, 90%, 95%, 98%, or 99% identical to a nucleic acid sequence represented in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15. Furthermore, the invention contemplates that exemplary antagonistic variants are encoded by a nucleic acid sequence that hybridizes under stringent conditions, including a wash step of 0.2×SSC at 65° C., to at least one of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15.

This application also describes methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by products. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids. For example, such methods are used to produce fusion proteins including domains which facilitate purification or immunodetection, and to produce recombinant mutant forms of a protein (for example a dominant negative or other antagonistic variant of an enzyme that catalyzes GAG chain initiation, GAG chain elongation, or GAG chain sulfation).

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors. For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, p SV2gpt, p SV2neo, p SV2-dhfr, p Tk2, p RSVneo, p MSG, p SVT7, pko-neo, pBacMam-2, and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the β-gal containing pBlueBac III).

When it is desirable to express only a portion of a protein, such as a form lacking a portion of the N-terminus, e.g. a truncation mutant, it may be necessary to add a start codon (ATG) to the oligonucleotide fragment containing the desired sequence to be expressed. It is well known in the art that a methionine at the N-terminal position can be enzymatically cleaved by the enzyme methionine aminopeptidase (MAP).

Techniques for making fusion genes are known to those skilled in the art. The joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another example, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence.

The present invention also makes available isolated polypeptides which are isolated from, or otherwise substantially free of other cellular and extracellular proteins. The term “substantially free of other cellular or extracellular proteins” (also referred to herein as “contaminating proteins”) or “substantially pure or purified preparations” are defined as encompassing preparations having less than 20% (by dry weight) contaminating protein, and preferably having less than 5% contaminating protein. Functional forms of the subject polypeptides can be prepared as purified preparations by using a cloned gene as described herein. By “purified”, it is meant, when referring to peptide or nucleic acid sequences, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water and buffers can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry.

The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo).

For example, it is reasonable to expect that, in some instances, an isolated replacement of a leucine with an isoleucine or valine, an aspartate with a glutamate, a threonine with a serine, or a similar replacement of an amino acid with a structurally related amino acid (e.g., isosteric and/or isoelectric mutations) may not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 5th ed. by Berg, Tymoczko and Stryer, WH Freeman and Co.: 2002). Whether a change in the amino acid sequence of a peptide results in a variant which maintains the same function as the wildtype protein, or a variant which antagonizes the function of the wildtype protein, can be determined by assessing the ability of the variant peptide to produce a response in cells in a fashion similar to the wild-type protein, or antagonize such a response. Polypeptides in which more than one replacement has taken place can readily be tested in the same manner.

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant functions as an antagonist, or whether a given variant has the same or substantially the same function as the wildtype protein. In the context of the present invention, several methods for assaying the functional activity of potential variants are provided.

To further illustrate, the invention contemplates a method for generating sets of combinatorial mutants, as well as truncation mutants, and is especially useful for identifying potential antagonistic variant sequences. The purpose of screening such combinatorial libraries is to generate, for example, novel variants which can antagonize the function of a GAG chain initiation enzyme, a GAG chain elongation enzyme, or a GAG chain sulfation enzyme.

In one aspect of this method, the amino acid sequences for a population of enzymes are aligned, preferably to promote the highest homology possible. By a population of proteins is meant the alignment of, for example, XT-I proteins from several different species (e.g., human, mouse, rat, etc.). Similarly the amino acid sequences of XT-II proteins, acetylgalactosaminyltransferase proteins, glucuronyltransferase proteins, or the amino acid sequences of any protein that catalyzes GAG chain initiation, GAG chain elongation, or GAG chain sulfation, from several different species could be aligned. Amino acids which appear at each position of the aligned sequences are selected to create a degenerate set of combinatorial sequences. In one example, the variegated library of variants is generated by combinatorial mutagenesis at the nucleic acid level, and is encoded by a variegated gene library. For instance, a mixture of synthetic oligonucleotides can be enzymatically ligated into gene sequences such that the degenerate set of potential sequences are expressible as individual polypeptides, or alternatively, as a set of larger fusion proteins (e.g. for phage display) containing the set of sequences therein.

The library of potential variants can be generated from a degenerate oligonucleotide sequence using a variety of methods. Chemical synthesis of a degenerate gene sequence can be carried out in an automatic DNA synthesizer, and the synthetic genes then ligated into an appropriate expression vector. One purpose of a degenerate set of genes is to provide, in one mixture, all the sequences encoding the desired set of potential variant sequences. The synthesis of degenerate oligonucleotides is known in the art.

A range of techniques are known for screening gene products of combinatorial libraries made by point mutations, and for screening cDNA libraries for gene products having a certain property. Such techniques will be generally adaptable for rapid screening of the gene libraries generated by the combinatorial mutagenesis of enzymes that catalyze GAG chain initiation, GAG chain elongation, and GAG chain sulfation. These techniques are also applicable for rapid screening of other gene libraries. One example of the techniques used for screening large gene libraries includes cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

The application also describes reducing a protein to generate mimetics, e.g. peptide or non-peptide agents. Mimetics having a desired biological activity can be readily tested in vitro or in vivo.

The present invention also contemplates the use of nucleic acid inhibitors such as antisense oligonucleotide, RNAi constructs, DNA enzymes, and ribozymes. The selection of optimal nucleic acid sequences to inhibit the function and/or activity of one or more enzymes that catalyze GAG chain initiation, GAG chain elongation, or GAG chain sulfation (e.g., sequences that inhibit GAG chain biosynthesis) can be facilitated by the construction and screening of libraries of nucleic acid sequences following similar methodology as outlined in detail above.

Similarly, the present invention also contemplates the use of small organic molecules that inhibit the function and/or activity of one or more enzymes that catalyze GAG chain initiation, GAG chain elongation, or GAG chain sulfation. A variety of chemical libraries and libraries of small organic molecules are available, and these can be readily screening for agents with the desired activities.

Constructs comprising the subject agents may be administered in biologically effective carriers, e.g. any formulation or composition capable of effectively delivering the agents to cells in vivo or in vitro. The particular approach can be selected from amongst those well known to one of skill in the art based on the particular agent to be delivered (e.g., DNA enzyme, polypeptide variant, peptidomimetic, RNAi construct, antibody, antisense oligonucleotide, small organic molecule, and the like), the cell type to which delivery is desired, and the route of administration.

Approaches include viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors can be used to deliver nucleic acids, for example RNAi constructs, as well as to deliver nucleic acids encoding particular proteins such as polypeptide variants. These vectors provide efficient delivery of genes into cells. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-i (HSV-1). HSV-1 based vectors may be especially useful in the methods of the present invention because they have been previously shown to infect neuronal cells. Given that many adult neuronal cells are post-mitotic, and thus have been difficult to infect using some other commonly employed viruses, the use of HSV-1 represents a substantial advance and further underscores the potential utility of viral based systems to facilitate gene expression in the nervous system (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be administered by simply contacting cells with said protein. For example, cells are typically cultured in media which is supplemented by a number of proteins such as FGF, TGFβ, insulin, etc. These proteins influence cells by simply contacting the cells. Such a method similarly pertains to other agents such as small organic molecules and chemical compounds. These agents may either exert their effect at the cell surface, or may be able to permeate the cell membrane without the need for additional manipulation.

In another embodiment, a polypeptide, such as an antagonistic variant of an enzyme that catalyzes GAG chain initation, GAG chain elongation, or GAG chain sulfation, is directly introduced into a cell. Methods of directly introducing a polypeptide into a cell include, but are not limited to, protein transduction and protein therapy. For example, a protein transduction domain (PTD) can be fused to a nucleic acid encoding a particular polypeptide antagonist, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

(v) Models of Glial Scar Formation and Neuronal Regeneration

A variety of in vitro and in vivo models are known in the art, and can be used to test the efficacy of a particular agent. Exemplary agents for use in the methods of the present invention include agents with one or more of the following functional properties: (a) decrease glial scar formation, (b) decrease GAG content in a glial scar, (c) decrease expression of one or more proteoglycans expressed in a glial scar, (d) promote inter-mixing of Schwann cells and astrocytes, or (e) promote neuronal regeneration. Accordingly, exemplary in vitro and in vivo models allow one of skill in the art to assay the ability of an agent to perform at least one of these functions.

In vitro assays include cell-free assays. For example, the ability of a given agent to inhibit the activity of a given protein can be assayed in a cell free system using purified proteins. Further cell free assays include binding assays to verify that a given agent interacts directly with a protein, such as a protein that catalyzes GAG chain initiation, GAG chain elongation, or GAG chain sulfation, or a proteoglycan that is expressed in a glial scar.

In addition to cell-free assays, exemplary in vitro assays include cell-based assays. For example, the ability of a given agent to inhibit expression and/or activity of an enzyme that catalyzes GAG chain initiation, GAG chain elongation, or GAG chain sulfation can be measured in any number of primary cell cultures or cell lines. Suitable cells include, without limitation, primary neuronal cells, transformed neuronal cell lines, and fibroblasts. Furthermore, given the high content of GAG in cartilage, primary cartilage and chondrocyte cultures, as well chondrocytic and other mesenchymal cell lines, such cells are also suitable for assaying the efficacy of an agent.

To further mimic the cell damage which results in reative gliosis, cells in culture can be subjected to injury or damage prior to the testing of the agent. For example, cells can be cultured in the presence of a toxin, a protease, or other chemical, or cells can be physically damaged such as by exposure to suboptimal temperature or oxygen conditions, by scraping, or by manual tituration.

Further suitable cell based assays are described in the examples. One such model which mimics, at least in part, some of the cellular consequences of a glial scar is the Schwann cell-astrocyte confrontation assay. Briefly, under standard in vitro culture conditions, Schwann cells and astrocytes grown in co-culture do not intermingle. Both cell types will grow in culture until they contact the other cell type, and at that point the cells will grow in a restricted matter and will not intermingle with the other cell type. Analysis of the border between the Schwann cells and the astrocytes reveals extensive expression of GAGs, and this high GAG content at the border between the Schwann cells and the astrocytes is hypothesized to play a role in restricting the inter-mixing between these cell populations. As used herein, the terms “inter-mixing” and “intermingle” are used interchangeably and denote a scenario that is a typical for Schwann cells and astrocytes grown in co-culture. The more typical scenario (the opposite of inter-mix) with respect to the interaction between Schwann cells and astrocytes grown in co-culture is that each cell type grows in a segregated fashion with other like cells and the c ells respect the boundary between their two cell types. In other words, typically, Schwann cells will grow together with other Schwann cells and astrocytes with grow together with other a strocytes, and cells will generally respect the border between the population of Schwann cells and the population of astrocytes.

Given that Schwann cells and astrocytes typically fail to inter-mix, and that the border between the two cell types is characterized by extensive GAG expression, this co-culture system represents a suitable in vitro screening assay for identifying and characterizing agents that decrease GAG content and or promote inter-mixing of Schwann cells and astrocytes. Such agents are candidate agents for promoting neuronal regeneration, and their ability to promote neuronal regeneration can be verified in a secondary assay.

In vivo assays can be conducted in any suitable animal model, such as an on-human animal model. Exemplary non-human species in which such experiments may be conducted include vertebrates such as amphibians, birds, reptiles, fish, and mammals. Exemplary non-human mammals include mice, rats, rabbits, cats, dogs, goats, sheep, pigs, and non-human primates. At some point, suitable testing of agents whose safety and efficacy has been demonstrated in a number of non-human animal models may be conducted in humans.

Agents can be tested in animal models of neuronal injury. Such animal models of neuronal injury include animals with damage to the spinal cord, animals with an injury to the brain, animals with an injury to the retina, animals with an injury to the sciatic nerve, and animals with an injury to peripheral nervous tissue. Such injuries may be caused in any of a variety of ways including, but not limited to, by severing, crushing, breaking, burning, freezing, or contacting with a chemical or infectious agent.

Agents can also be tested in an animal model of a neurodegenerative disease. Such animal disease models may be due to naturally occurring genetic lesions, may be engineered through knockout or transgenic manipulations, or may be induced by chemical, bacterial, viral, or other non-genetic means. Exemplary animals models include animal models of Parkinson's disease, Huntington's disease, Alzheimer's disease, ALS, peripheral neuropathy, diabetic neuropathy, and macular degeneration.

(vi) Method of Screening

This application describes methods and compositions that inhibit the expression and/or activity of one or more enzymes that catalyze GAG chain initiation, GAG chain elongation, or GAG chain sulfation. This application further describes methods and compositions that inhibit the expression of proteoglycan that comprise a glial scar. This application further describes methods and compositions for promoting inter-mixing of Schwann cells and astrocytes. This application still further describes methods and compositions that promote neuronal regeneration. Exemplary agents (e.g., agents which inhibit an enzyme that catalyzes GAG chain initiation, GAG chain elongation, GAG chain sulfation, or agents that inhibit expression of one or more proteoglycan that comprises a glial scar) have one or more of the following activities: decrease glial scar formation, decrease GAG content in a glial scar, decrease proteoglycan expression in a glial scar, promote inter-mixing of Schwann cells and astrocytes, or promote neuronal regeneration. The present invention further contemplates methods of identifying additional agents which possess one or more of these functions. These five activities that characterize the agents of the present invention will also be referred to herein as “desired neuronal activity”. Exemplary agents promote neuronal regeneration when administered in an effective amount.

Agents screened (e.g., a single agent, a combination of two or more agents, a library of agents) include nucleic acids, peptides, proteins, antibodies, antisense RNAs, RNAi constructs (including siRNAs), DNA enzymes, ribozymes, morpholino constructs, chemical compounds, and small organic molecules. Agents may be screened individually, in combination, or as a library of agents.

Without being bound by theory, an agent identified by the subject methods as having one or more of the desired neuronal activities may work via any one of a number of mechanisms. In one example, such agents inhibit the expression and/or activity of an enzyme that catalyzes GAG chain initiation, such as a xylosyltransferase. Exemplary xylosyltransferases include XT-I and XT-II. In another example, such agents inhibit the expression and/or activity of an enzyme that catalyzes GAG chain elongation, such as an acetylgalactosaminyltransfease, a glucuronyltransferase, or a glucoaminotransferase. Exemplary enzymes include N-acetylgalactosaminyltransferase and glucuronyltransferase. In another example, such agents inhibit the expression of a proteoglycan that comprises a glial scar. Exemplary proteoglycans that comprise a glial scar include neurocan, NG2, and phosphacan.

In addition to being directed to the inhibition of one or more of a variety of enzymes and proteins involved in glial scar formation, the invention contemplates that agents identified as having one or more of the desired neuronal activities may exert their inhibitory effect on the expression and/or activity of that protein in any one of a number of ways.

In many drug screening programs that test libraries of nucleic acids, polypeptides, chemical compounds and natural extracts, high throughput assays are desirable to increase the number of agents surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target which is mediated by a test agent. Cell free systems include in vitro systems (preparations of proteins and agents combined in a test tube, Petri dish, etc.), as well as cell free systems such as those prepared from egg extracts or reticulocyte lysates. Moreover, the effects of cellular toxicity and/or bioavailability of the test agents can be generally ignored in such a system, the assay instead being focused primarily on the effect of the agent.

A primary screen can be used to narrow down agents that are more likely to have an effect on neuronal regeneration, in vitro and/or in vivo. Such a cell free system for use in the present invention may include a biochemical assay measuring activity one or more enzyme which catalyzes GAG chain initiation, GAG chain elongation, or GAG chain sulfation. To further illustrate for example, an XT-I polypeptide may be contacted with one or more agents (e.g., individual candidate agents, combinations of two or more agents, a library of nucleic acids, polypeptides, small organic molecules, chemical compounds, etc.) and the ability of the agent to decrease the activity of the XT-I polypeptide can be measured. The activity of the XT-I polypeptide can be assessed by comparing the ability of the XT-I polypeptide to modify a model substrate. One or more agents which decrease the ability of the XT-I polypeptide to modify a target substrate, in comparison to the ability of the XT-I polypeptide to modify the target substrate in the absence of the one or more agents, is a candidate agent for use in the subject methods. Similarly, an XT-II polypeptide may be contacted with one or more agents (e.g., individual candidate agents, combinations of two or more agents, a library of nucleic acids, polypeptides, small organic molecules, chemical compounds, etc.) and the ability of the agent to decrease the activity of the XT-II polypeptide can be measured. The activity of the XT-II polypeptide can be assessed by comparing the ability of the XT-II polypeptide to modify a model substrate. One or more agents which decrease the ability of the XT-II polypeptide to modify a target substrate, in comparison to the ability of the XT-II polypeptide to modify the target substrate in the absence of the one or more agents, is a candidate agent for use in the subject methods. Similarly, the ability of an agent to inhibit the activity of any enzyme that catalyzes GAG chain initiation, GAG chain elongation, or GAG chain sulfation can be examined using this type of methodology.

The efficacy of the agent can be assessed by generating dose response curves from data obtained using various concentrations of the test agent. Moreover, a control assay can also be performed to provide a baseline for comparison. Such candidates can be further tested for efficacy in promoting extension of cellular processes in neuronal cells in vitro, for efficacy in decreasing the activity and/or expression of the particular enzyme in other assays, for efficacy in neuronal regeneration in vitro or in vivo, for efficacy in decreasing glial scar formation, or for efficacy in decreasing glycosylaminoglycan modifications (GAGs) in vitro or in vivo.

In addition to cell-free assays, such as described above, the invention further contemplates the generation of cell-based assays for identifying agents having one or more of the desired neuronal activities. Cell-based assays may be performed as either a primary screen, or as a secondary screen to confirm the activity of agents identified in a cell free screen, as outlined in detail above. Such cell based assays can employ any cell-type. Exemplary cell types include neuronal cell lines, primary neural cultures, fibroblasts, lymphocytes, mesenchymal cells, etc. Cells in culture are contacted with one or more agents, and the ability of the one or more agents to promote the extension of cellular processes is measured. Agents which promote the extension of cellular processes are candidate agents for use in the subject methods of promoting neuronal regeneration.

In addition to assessing the ability of the test agent to promote neurite extension in cells in culture, the ability of the test agent to decrease glial scar formation, to decrease GAG contend, or to decrease the expression of one or more proteoglycan that comprise a glial scar can also be assessed. As with any screen, the particular phenotype is measured and compared in the presence or absence of the test agent.

A further example of a cell-based assay for identifying agents having one or more of the desired neuronal activities are based on assays which mimic some of the inhibitory characteristics of a glial scar. For example, cells in culture can be subjected to cellular damage such as by scraping, or exposure to a chemical or toxic agent.

As detailed herein, another suitable cell-based assay for identifying and characterizing agents for use in the subject methods is a Schwann cell-astrocyte confrontation assay. Agents (e.g., individual agents, combinations of agents, libraries of agents) can be screened and assayed for their ability to promote inter-mixing of Schwann cells and astrocytes grown in co-culture. The degree of inter-mixing can be readily evaluated by comparing co-cultures of cells grown in the presence versus the absence of test agent(s). Additionally, either in combination with an assessment of the degree of inter-mixing or independent of such an assessment, the effect of an agent on a confrontation co-culture can be measured by analyzing GAG expression at the Schwann cell-astrocyte interface. Candidate agents for use in the methods of the present invention would decrease GAG expression at the Schwann cell-astrocyte interface and/or promote inter-mixing of Schwann cells and astrocytes. The significance of any results obtained through the use of this assay would be determined by comparing the co-culture of cells in the presence versus the absence of the agent.

One class of agents that may inhibit the activity and/or expression of an enzyme that catalyzes GAG initiation, elongation, or sulfation are agents which bind directly to the mRNA encoding the particular enzyme. Exemplary agents which may inhibit the activity and/or expression of an enzyme that catalyzes GAG initiation, GAG elongation, or GAG sulfation by binding directly to the mRNA encoding such the enzyme include antisense oligonucleotides, RNAi oligonucleotides, ribozymes, and DNA enzymes. Another class of agent that may inhibit the activity and/or expression of an enzyme that catalyzes GAG initiation, elongation, or sulfation are agents which bind directly to that particular enzyme. Exemplary agents which may inhibit the activity and/or expression of an enzyme that catalyzes GAG chain initiation, GAG chain elongation, or GAG chain sulfation by binding directly to the protein include antibodies, small organic molecules, antagonistic variants of the wildtype enzyme, and antagonistic fragments of the wildtype enzyme.

Accordingly, the present invention contemplates screening for agents which bind to, either directly or indirectly, mRNA encoding an enzyme that catalyzes GAG chain initiation, GAG chain elongation, or GAG chain sulfation, as well as screening for agents which bind to, either directly or indirectly, a protein that catalyzes GAG chain initiation, GAG chain elongation, or GAG chain sulfation.

Many methods exist in the art for assessing the interactions between proteins, nucleic acids, small organic molecules, antibodies, and the like. Exemplary methods include two-hybrid screens, affinity chromatography, immunoprecipitation, nucleic acid hybridization, and the like. One of skill in the art can select among commonly used methods for detecting the interaction between the mRNA or protein corresponding to a particular enzyme that catalyzes GAG chain initiation, GAG chain elongation, or GAG chain sulfation and a given agent such as a protein, nucleic acid, antibody, antisense oligonucleotide, DNA enzymes, small organic molecule, RNAi construct, ribozyme, and the like.

In addition to the cell free and cell based assays described above. Agents may be screened in vivo using animal models of neuronal injury and/or neural degeneration. Exemplary agents can be identified by any of the following characteristics: (i) the ability to decrease expression and/or activity of an enzyme that catalyzes GAG chain initiation, GAG chain elongation, or GAG chain sulfation in the animal model, (ii) the ability to decease expression of a proteoglycan that comprises a glial scar in the animal model, (iii) the ability to decrease glial scar formation, (iv) the ability to decrease GAG content in a glial scar in the animal model, (v) the ability to promote inter-mixing of Schwann cells and astrocytes, and/or (vi) the ability to promote neuronal regeneration in the animal.

The foregoing represent illustrative examples of assays that can be used to identify and characterize agents for use in the subject methods. Other aspects of the invention provide for certain methods of further analyzing, developing, and marketing candidate agents which demonstrate the ability to (i) decrease expression and/or activity of an enzyme that catalyzes GAG chain initiation, GAG chain elongation, or GAG chain sulfation, (ii) decrease expression of a proteoglycan that comprises a glial scar, (iii) decrease glial scar formation, (iv) promote inter-mixing of Schwann cells and astrocytes, and/or (v) promote neuronal regeneration. Of particular interest is the identification, characterization, and development of an agent(s) that promote neuronal regeneration.

In particular, practicing any of a variety of assay methods, as exemplified herein, may identify certain agents that have one or more of the functions of the present invention. This technical step, when combined with one of more additional steps, provides pharmaceutical compositions which can be developed, tested, approved for use in humans, marketed, and sold. For example, agents according to the present invention can be tested for efficacy as therapeutics in a variety of disease models, and the potential therapeutic compositions can then be tested for toxicity and other safety-profiling before formulating, packaging and subsequently marketing the resulting formulation for the treatment of disease. Alternatively, the rights to develop and market such formulations or to conduct such steps may be licensed to a third party for consideration. In certain other aspects of the invention, the agents thus identified may have utility in the form of information that can be provided to a third party for consideration such that an improved understanding of the function or side effects of said agent in a biological or therapeutic context is obtained, or to provide an improved understanding of the cellular mechanisms that inhibit neuronal regeneration in vivo.

In certain embodiments, the initially identified agent can be subjected to further optimization, e.g., to further refine the structure of a lead agent. Such optimization may lead to the development of analogs (e.g., modified versions of the originally identified agent) that maximize the desirable pharmacological characteristics including: solubility, permeability, bioavailability, toxicity, mutagenicity, and pharmacokinetics.

Structural modifications are made to a lead analog to address issues with the parameters listed above. These modifications however, must take into account possible effects on the analog's potency and activity. For example, if the toxicity of a lead analog is high when tested in an animal model, modifications can be made to the analog in an effort to decrease toxicity while maintaining the desired characteristic of promoting neuronal regeneration.

Candidate agents (whether or not said agent is modified to make an analog of the originally identified agent possessing improved in vivo characteristics) or combinations thereof must be tested for efficacy and toxicity in animal models. Such therapeutic profiling is commonly employed in the pharmaceutical arts. Before testing an experimental therapeutic in humans, extensive therapeutic profiling (preclinical testing) must be completed to establish initial parameters for safety and efficacy. Preclinical testing establishes a mechanism of action for the therapeutic, its bioavailability, absorption, distribution, metabolism, and elimination through studies performed in vitro (that is, in test tubes, beakers, petri dishes, etc.) and in animals. Animal studies are used to assess whether the therapeutic will provide the desired results. Varying doses of the experimental therapeutic are administered to test the therapeutic's efficacy, identify harmful side-effects that may occur, and evaluate toxicity.

Briefly, one of skill in the art will recognize that the identification of a candidate agent is a first step in developing a pharmaceutical preparation useful for administration. The agent must be formulated in a pharmaceutically acceptable carrier (e.g., a pharmaceutical preparation or pharmaceutical composition). Administration of a pharmaceutical preparation comprising said agent in an amount effective to treat a condition or disease must be both safe and effective. Early stage drug trials, routinely used in the art, help to address concerns of the safety and efficacy of a potential pharmaceutical. We have briefly outlined specific cell culture based and animal models which could be used in the early stages of evaluating lead compound. Following initial identification of lead agents, further animal studies are necessary before initiation of human trials. Briefly, mice or rats could be administered varying doses of said pharmaceutical preparations over various time schedules. The route of administration would be appropriately selected based on the particular characteristics of the agent and on the cell type to which delivery of the agent is desired. Control mice can be administered a placebo (e.g., carrier or excipient alone).

In one embodiment, the step of therapeutic profiling includes toxicity testing of agents in cell cultures and in animals; analysis of pharmacokinetics and metabolism of the candidate agent; and determination of efficacy in animal models of relevant diseases. In certain instances, as for example when the agent is a small organic molecule, the method can include analyzing structure-activity relationship and optimizing lead analogs based on efficacy, safety and pharmacokinetic profiles. The goal of such steps is the selection of agents, or analogs of the originally identified agent, for pre-clinical studies to lead to filing of Investigational New Drug applications (“IND”) with the FDA prior to human clinical trials.

Between lead optimization and therapeutic profiling, one goal is to develop an agent that maintains the desired biological effect (e.g., to promote neuronal regeneration) and can be administered with minimal side-effects. Exemplary agents should not be exceptionally toxic (e.g., should have only tolerable side-effects when administered to patients), should not be mutagenic, and should not be carcinogenic.

By toxicity profiling is meant the evaluation of potentially harmful side-effects which may occur when an effective amount of a pharmaceutical preparation is administered. A side-effect may or may not be harmful, and the determination of whether a side effect associated with a pharmaceutical preparation is an acceptable side effect is made by the Food and Drug Administration during the regulatory approval process. This determination does not follow hard and fast rules, and that which is considered an acceptable side effect varies due to factors including: (a) the severity of the condition being treated, (b) the availability of other treatments, and (c) the side-effects associated with these currently available treatments. Presently, there are few treatment options available for individuals suffering from a spinal cord injury. Similarly, there are few treatments that provide prolonged or permanent improvements for patients suffering from Parkinson's disease, macular degeneration, Alzheimer's disease, ALS, multiple sclerosis, and many other neurodegenerative diseases. Given the paucity of treatment options for such patients, it is likely that a certain spectrum of side-effects would be considered tolerable. This is contrast to other diseases or conditions which are either not life-threatening or for which other safe and effective treatments already exist. Under these circumstances, it is likely that fewer and less severe side-effects would be considered tolerable. Nevertheless, the goal of the production of any pharmaceutical product is to minimize the number and degree of side-effects associated with administration of the pharmaceutical preparation, while maximizing the therapeutic effect of that pharmaceutical preparation.

Toxicity tests can be conducted in tandem with efficacy tests, and mice administered effective doses of the pharmaceutical preparation can be monitored for adverse reactions to the preparation.

One or more agents, or analogs thereof, which are proven safe and effective in animal studies (both non-human and human), can be formulated into a pharmaceutical preparation, and following FDA approval, readied for sale. Such pharmaceutical preparations can then be marketed, distributed, and sold. Exemplary agents may be marketed and sold alone, or may be sold as a pharmaceutical package and/or kit. Such kits include the pharmaceutical preparation along with instructions for its use. Such kits may also include devices necessary for administration of the agent such as catheters, osmotic pumps, and the like.

Furthermore, in any of the foregoing aspects, the invention appreciates that a method of providing a pharmaceutical preparation for promoting neuronal regeneration does not necessarily end with the formulation and sale of a pharmaceutical product. Such a method may also include a system for billing a patient and/or a patient's insurance provider, as well as a system for collecting appropriate reimbursement from the patient and/or the patient's insurance provider.

(vii) Methods of Administration of Nucleic Acids, Proteins, Chemical Compounds and Pharmaceutical Compositions of Agents

An agent identified by the subject methods has many potential uses. Such an agent may be a nucleic acid, peptide, polypeptide, RNAi construct, chemical compound, small organic molecule, antisense RNA, ribozyme, DNA enzyme, morpholino construct, antibody, and the like. By agent is meant to include a single agent, or a combination of agents which together possess the desired activity. An exemplary agent inhibits the expression and/or activity of an enzyme that catalyzes GAG chain initiation, GAG chain elongation, GAG chain sulfation, or inhibits the expression of a proteoglycan that comprises a glial scar, and such agents have one or more of the following activities: (a) decrease glial scar formation, (b) decrease GAG content in a glial scar, (c) decrease expression of one or more proteoglycan expressed in a glial scar, (d) promote inter-mixing of Schwann cells and astrocytes, or (e) promote neuronal regeneration. In one embodiment, an agent promotes neuronal regeneration.

Agents which possess one of more of these characteristics may be useful in a therapeutic context. For example, glial scarring which occurs following an injury to the central nervous system inhibits neuronal regeneration. This phenomenon is thought to account for the minimal regeneration observed in the CNS following injury. Furthermore, this phenomenon is thought to account for the minimal improvement which occurs following onset of symptoms of a neurodegenerative disease. In addition to glial scarring, other injury or disease to the central or peripheral nervous system can impair the ability of the damaged neuronal tissue to regenerate following the injury. Impairment of regeneration in the central or peripheral nervous system may be due, for example, to inflammation, to a physical and/or molecular barrier which prevents regeneration, or to cellular damage which prevents neuronal cells from responding to local molecular, chemical and/or electrical cues.

A principle source of the inhibitory influence of glial scar tissue is thought to be the presence of proteoglycans including glycosaminoglycans (GAGs). Accordingly, agents which reduce the expression and/or activity of the enzymes responsible for GAG biosynthesis or agents which decrease the expression of proteoglycan that comprise a glial scar reduce the presence of GAGs at the site of injury, and the reduction of GAGs at the site of injury promotes regeneration of neuronal cells. Thus, the methods and compositions of the present invention provide novel treatment options to promote neuronal regeneration, and thus aid in recovery from injuries and degenerative diseases of the central and peripheral nervous system.

The application provides agents that inhibit expression and/or activity of one or more enzymes that catalyze GAG chain initiation, GAG chain elongation, or GAG chain initiation, as well as agents that decrease the expression of proteoglycans that comprise a glial scar. In a preferred embodiment, such agents promote neuronal regeneration. These agents may be used either alone, or may be used in combination with one or more agents that promote neuronal regeneration. Furthermore agents identified in the subject methods may be administered as part of a therapeutic regimen in combination with other agents such a neurotrophic factors, growth factors and/or enzymes which promote degradation of proteoglycan sugars in glial scars. Additionally, agents identified in the subject methods may be administered as part of a therapeutic regimen in combination with other methods used to treat the specific neuronal injury or neurodegenerative disease. For example, in the case of Parkinson's disease, a subject agent may be administered in combination with L-dopa or other Parkinson's disease medications, or in combination with a cell based neuronal transplantation therapy for Parkinson's disease. In the case of an injury to the brain or spinal cord, a subject agent may be administered in combination with physical therapy, hydrotherapy, massage therapy, and the like. In the case of peripheral neuropathy, as for example diabetic neuropathy, a subject agent may be administered in combination with insulin.

Agents that decrease glial scar formation, decrease GAG content in a glial scar, or decrease expression of proteoglycan that comprise a glial scar can promote neuronal regeneration, even in the presence of the proteoglycans which compose glial rich scar tissue. Accordingly, the methods of the present application can be used to overcome the inhibitory effects of glial rich scar tissue, and thus be used to promote neuronal regeneration following tissue injury and/or degeneration, including tissue injury or degeneration which results in formation of a glial rich scar. The methods of the present application thus provide a novel treatment option for patients afflicted with any of a number of conditions which result in injury or degeneration of neuronal cells of the central and peripheral nervous system. Examples of conditions which can be treated by the methods described herein include, without limitation, spinal cord injury, brain injury (following surgery, stroke, cancer treatment, or trauma), peripheral nerve injury, Parkinson's disease, Huntington's disease, detached retina, macular degeneration, Alzheimer's disease, amotrophic lateral sclerosis (ALS), multiple sclerosis, peripheral neuropathy, and diabetic neuropathy. One of skill in the art will appreciate that injuries or conditions that results in damage to or degeneration of neurons are candidates for treatment with the compositions of the present application.

Furthermore, the invention contemplates the identification of agents that promote neuronal regeneration in the absence of a glial scar. Such agents may be particularly useful for improving neuronal regeneration, even in conditions that are not characterized by glial scar formation.

Exemplary Conditions Which May be Treated by the Methods of the Present Invention.

a. Injury

A physical injury to cells of the CNS may result in glial scar formation which inhibits neuronal regeneration and thus interferes with recovery from the injury. Such injuries include physical injuries to cells of the CNS including cells of the brain, spinal cord and eye. Some physical injuries may not result in glial scar formation, but may still inhibit regeneration due to inflammation, trauma, and so on. Examples of physical injuries include, but are not limited to, crushing or severing of neuronal tissue, such as may occur following a fall, car accident, gun shot or stabbing wound, etc. Further examples of physical injuries include those caused by extremes in temperature such as burning, freezing, or exposure to rapid and large temperature shifts.

Further examples of an injury to cells of the CNS include those caused by infection such as by a bacterial or viral infection. Examples of bacterial or viral infections affecting cells of the CNS include meningitis, staph, and HIV. However, one of skill in the art will recognize that many different types of bacteria or viruses may infect cells of the CNS and cause injury.

Additionally, injury to cells of the CNS often occurs as a consequence or side effect of other treatments being used to relieve the effects of a condition of the CNS. For example, individuals often undergo surgery to relieve discomfort, numbness, and/or lack of mobility which results from a pinched nerve, bulging disk, etc. Such surgery may result in additional injury. Additional examples of surgeries involving cells of the CNS include brain surgery to relieve intracranial pressure, surgery to remove a malignant or benign tumor, surgery to treat an anuryism, surgery to insert a stent, intraluminal device, implant, etc. In the foregoing examples, injury following surgery may be the result of error on the part of the physician, or may be a normal side effect of the successful surgical procedure.

Other treatment regimens which may cause injury to cells of the CNS include cancer therapies. Chemotherapeutic agents, radiation therapy, and the like may do substantial injury not only to cancerous cells, but also to healthy cells.

In addition to injuries to cells of the central nervous system, injury of cells of the peripheral nervous system may result in the formation of scar tissue, inflammation, trauma, and the like, and such mechanisms may inhibit regeneration. Accordingly, the present invention contemplates that the subject agents can be used to promote neuronal regeneration, including increase the rate and/or extent of neuronal regeneration, in neuronal cells of the central and peripheral nervous system.

b. Neurodegenerative Diseases

A wide range of neurodegenerative diseases cause extensive cell damage (i.e., injury) to cells of the CNS and PNS. Accordingly, neurodegenerative diseases are candidates for treatment using the described agents. Administration of a subject agent can promote neuronal regeneration in the CNS or PNS of a patient with a neurodegenerative disease, and the promotion of neuronal regeneration can ameliorate, at least in part, symptoms of the disease. Agents may be administered individually, in combination with other agents of the invention, or as part of a treatment regimen appropriate for the specific condition being treated. The following are illustrative examples of neurodegenerative conditions which can be treated using the subject agents.

Parkinson's disease is the result of the destruction of dopamine-producing neurons of the substantia nigra, and results in the degeneration of axons in the caudate nucleus and the putamen degenerate. Although therapies such as L-dopa exist to try to ameliorate the symptoms of Parkinson's disease, to date we are unaware of treatments which either prevent the degeneration of axons and/or increase neuronal regeneration. Administration of agents with promote neuronal regeneration can help to ameliorate at least certian symptoms of Parkinson's disease including rigidity, tremor, bradykinesia, poor balance and walking problems.

Alzheimer's disease, a debilitating disease characterized by amyloid plaques and neurofibrillary tangles, results in a loss of nerve cells in areas of the brain that are vital to memory and other mental abilities. There also are lower levels of chemicals in the brain that carry complex messages back and forth between nerve cells. Alzheimer's disease disrupts normal thinking and memory. The incidence of Alzheimer's disease will only increase as the average life expectancy continues to rise around the world. One of the most notable features of Alzheimer's disease is that affected individuals can live for extended periods of time (ten or more years) while being in an extremely debilitated state often requiring round the clock care. Accordingly, the disease takes not only an enormous emotional toll, but also exacts a tremendous financial toll on affected individuals and their families. Therapies which improve neuronal function, for example neurite outgrowth, have substantial utility in improving the quality of life of Alzheimer's sufferers.

Huntington's disease is a degenerative disease whose symptoms are caused by the loss of cells in a part of the brain called the basal ganglia. This cell damage affects cognitive ability (thinking, judgment, memory), movement, and emotional control. Symptoms appear gradually, usually in midlife, between the ages of 30 and 50. However, the disease can also strike young children and the elderly. Huntington's disease is a genetic disorder. Although people diagnosed with the disease can often maintain their independence for several years following diagnosis, the disease is degenerative and eventually fatal. Currently, there are no treatments available to either cure or to ameliorate the symptoms of this disease. Furthermore, the onset of Huntington's disease is typically in middle-age (approx age 40), at a time when many people have already had children. Thus, people have usually passed this fatal genetic disorder to their off-spring before they realize that they are ill.

Amyotrophic lateral sclerosis (ALS), often referred to as “Lou Gehrig's disease,” is a progressive neurodegenerative disease that attacks motor nerve cells in the brain and the spinal cord. Degeneration of motor neurons affect the ability of the brain to initiate and control muscle movement. With all voluntary muscle action affected, patients in the later stages of the disease become totally paralyzed, and eventually die.

Multiple sclerosis (MS) is an illness diagnosed in over 350,000 persons in the United States today. MS is characterized by the appearance of more than one (multiple) areas of inflammation and scarring of the myelin in the brain and spinal cord. Thus, a person with MS experiences varying degrees of neurological impairment depending on the location and extent of the scarring. The most common characteristics of MS include fatigue, weakness, spasticity, balance problems, bladder and bowel problems, numbness, vision loss, tremor and vertigo. The specific symptoms, as well as the severity of these symptoms, varies from patient to patient and is largely determined by the particular location within the brain of the lesions.

MS is considered an autoimmune disease. Recent data suggest that common viruses may play a role in the onset of MS. If so, MS may be caused by a persistent viral infection or alternatively, by an immune process initiated by a transient viral infection in the central nervous system or elsewhere in the body. Epidemiological studies indicating the distribution of MS patients suggest that there is at triggering factor responsible for initiating onset of the disease. Without being bound by theory, tt appears that some environmental factor, most likely infectious, must be encountered.

The incidence of MS is higher in North America and Europe and this geographic distribution is further suggestive of an environmental influence(s) underlying onset of MS. Additionally, MS is more prevalent in women than in men, and is more common amongst Caucasians than within either Hispanic or African-American populations. Interestingly, MS is extremely rare within Asian populations. Macular degeneration is a catch-all term for a number of different disorders that have a common end result: the light-sensing cells of the central region of the retina—the macula—malfunction and eventually die, with gradual decline and loss of central vision, while peripheral vision is retained. Most cases of macular degeneration are isolated, individual, occurrences, mostly in people over age 60. These types are called Age Related Macular Degeneration (AMD). More rarely however, younger people, including infants and young children, develop macular degeneration, and they do so in clusters within families. These types of macular degeneration are collectively called Juvenile Macular Degeneration and include Stargardt's disease, Best's vitelliform macular dystrophy, Doyne's honeycomb retinal dystrophy, Sorsby's fundus dystrophy, Malattia levintinese, Fundus flavimaculatus, and Autosomal dominant hemorrhagic macular dystrophy.

Agents for use in the methods of the present invention, as well as agents identified by the subject methods may be conveniently formulated for administration with a biologically acceptable medium, such as water, buffered saline, polyol (for example, glycerol, propylene glycol, liquid polyethylene glycol and the like) or suitable mixtures thereof. Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Methods of introduction may also be provided by rechargeable or biodegradable devices. Various slow release polymeric devices have been developed and tested in vivo in recent years for the controlled delivery of agents, including proteinacious biopharmaceuticals. A variety of biocompatible polymers (including hydrogels), including both biodegradable and non-degradable polymers, can be used to form an implant for the sustained release of an agent at a particular target site. Delivery of agents to injury site can be attained by vascular administration via liposomal or polymeric nano- or micro-particles; slow-release vehicles implanted at the site of injury or damage; osmotic pumps implanted to deliver at the site of injury or damage; injection of agents at the site of injury or damage directly or via catheters or controlled release devices; injection into the cerebro-spinal fluid.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

One or more agents may be administered to humans and other animals by any suitable route of administration. With regard to administration of agents to the brain, it is known in the art that the delivery of agents to the brain may be complicated due to the blood brain barrier (BBB). Accordingly, the application contemplates that agents may be administered directly to the brain cavity. For example, agents can be administered intrathecally or intraventricularly. Administration may be, for example, by direct injection, by delivery via a catheter or osmotic pump, or by injection into the cerebrospinal fluid.

However, although the BBB may present an impediment to the delivery of agents to the brain, it is also recognized that many agents, including nucleic acids, polypeptides and small organic molecules, are able to cross the BBB following systemic delivery. Therefore, the current application contemplates that agents may be delivered either directly to the sight of injury in the CNS or PNS, or may be delivered systemically.

Actual dosage levels of the one or more agents may be varied so as to obtain an amount of the active ingredient which is effective to achieve a response in an animal. The actual effective amount can be determined by one of skill in the art using routine experimentation and may vary by mode of administration. Further, the effective amount may vary according to a variety of factors include the size, age and gender of the individual being treated. Additionally the severity of the condition being treated, as well as the presence or absence of other components to the individuals treatment regimen will influence the actual dosage. The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. Such additional compounds may include factors known to influence the proliferation, differentiation or migration of neuronal cells. These additional compounds may be administered sequentially to or simultaneously with the agents for use in the methods of the present invention. Exemplary compounds known to influence neuronal cell behavior include neurotrophic factors or growth factors known to promote cell survival. Such neurotrophic factors or growth factors include, without limitation, nerve growth factor (NGF), basic fibroblast growth factor (bFGF), brain-derived growth factor (BDGF), neurotrophin 3 (NT-3), neurotrophin 4 (NT-4), neurotrophin 5 (NT-5), glial derived neurotrophic factor (GDNF), and ciliary neurotrophic factor (CNTF).

In addition to the administration of one or more of the subject agents, optionally in combination with one or more neurotrophic factors or growth factors, the invention further contemplates that one or more of the subject agents can be optionally administered in combination with one or more enzymes which digest proteoglycans. Such enzymes act on the proteoglycans which comprise the glial scar, and help to decrease the physical and/or molecular barrier imposed by this glial scar.

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Furthermore, the invention contemplates that agents can be administered in combination with cellular therapies. For example, certain methods of treating neurodegenerative diseases are based on administration of exogenous cells to help compensate for the lost or damaged tissue. Such cells include stem cells, differentiated neuronal cells, and the like. Certain other methods of treating lesions in the CNS, for example lesions caused by a spinal cord injury, rely on the transplantation of Schwann cells or other support cells of the nervous system: Transplantation of such cells is thought to reduce cavitation that occurs following CNS lesions and may act as a cellular bridge to facilitate neuronal regeneration. In the context of the present invention, administration of the subject agents can be in combination with a cell-based therapy. Exemplary cell-based therapies include transplantation of Schwann cells. Further exemplary cell based therapies include transplantation of other support cells of the nervous system such as astrocytes. Still further exemplary cell based therapies include transplantation of embryonic stem cells; adult stem cells including, but not limited to neuronal stem cells; and differentiated neuronal cells.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the central nervous system, for example, intrathecal, intraventricular, intraspinal, or intracerebrospinal administration (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension; (4) topical application, for example, as a cream, ointment or spray applied to the skin; or (5) opthalamic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention. These are particularly useful for injury and degenerative disorders of the eye including retinal detachment and macular degeneration.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Design of Antisense Oligonucleotides that Inhibit Expression and/or Activity of XT-I.

To design suitable antisense oligonucleotides to XT-I, we examined the nucleic acid sequence of mouse, rat and human XT-I. The nucleic acid sequence encoding rat XT-I is provided in SEQ ID NO: 5, the nucleic acid sequence encoding human XT-I is provided in SEQ ID NO: 1, and the nucleic acid sequence encoding mouse XT-I is provided in SEQ ID NO: 9.

To design an antisense oligonucleotide specific for XT-I, we used a sequence in the C-terminus of the protein. The sequence (CAAGGTCGCAAGTCCCCCCA) corresponds to nucleotide 1771 to 1790 of the rat sequence provided in SEQ ID NO: 5.

An antisense oligonucleotide was designed based on this sequence. The antisense oligonucleotide has the sequence, from 5′ to 3′, TGGGGGGACTTGCGACCTTG (SEQ ID NO: 37). To increase the stability of the oligonucleotide, and help prevent degradation, we modified two nucleotides on each end of the antisense oligonucleotide. Specifically, these nucleotides were phosphothioated.

### Example 2

### Design of Antisense Oligonucleotides that Inhibit Expression and/or Activity of XT-I and XT-II.

To design suitable antisense oligonucleotides which recognize both XT-I and XT-II, we examined the nucleic acid sequence of mouse, rat and human XT-I and XT-II. To design an antisense oligonucleotide which recognizes both XT-I and XT-II, we used a sequence in the C-terminus of the protein. The sequence (CACATGTGGCGCCTGGG) corresponds to nucleotides 1024-1040 of the human XT-I sequence provided in SEQ ID NO: 1, nucleotides 1016-1032 of the rat XT-I sequence provided in SEQ ID NO: 5, nucleotides 1247-1263 of the human XT-II sequence provided in SEQ ID NO: 3, nucleotides 1141-1157 of the rat XT-II sequence provided in SEQ ID NO: 7.

An antisense oligonucleotide was designed based on this sequence. The antisense oligonucleotide has the sequence, from 5′ to 3′, CCCAGGCGCCACATGTG (SEQ ID NO: 38). To increase the stability of the oligonucleotide, and help prevent degradation, we modified two nucleotides on each end of the antisense oligonucleotide. Specifically, these nucleotides were phosphothioated.

### Example 3

### XT-I Antisense Oligonucleotides Decrease Extracellular Matrix Content

To test the efficacy of the XT-I antisense oligonucleotide described in Example 1, we assayed the ability of the XT-I antisense oligonucleotide to decrease GAG content in cortical astrocytes in culture. Our results indicate that the antisense oligonucleotide effectively inhibits expression of XT-I, and decreases GAG content in cells in culture.

Briefly, astrocytes were prepared from the cortex of newborn rats. Cortical astrocytes were plated on poly L-lysine coated glass coverslips. From previous experiments using cortical a strocytes, we had learned that a strocytes require extensive ECM deposition in order to form a confluent monolayer. If ECM deposition is compromised, the astrocytes form large cavities in culture. This phenomenon can be used as a primary assay to demonstrate that a particular agent, such as an XT-I antisense oligonucleotide, decreases ECM matrix deposition in these cells.

Cortical astrocytes were plated and cultured in the presence of XT-I antisense oligonucleotide or a control oligonucleotide. In the presence of XT-I oligonucleotide, but not the control oligonucleotide, the astrocytes formed extensive cavities indicative of a decrease in ECM deposition.

### Example 4

### XT-I Antisense Oligonucleotides Decrease GAG Content

The results summarized in Example 3 demonstrate the efficacy of XT-I antisense oligonucleotides. To further examine the effects of these oligonucleotides, as well as other agents, we developed a second assay using cultures of cortical astrocytes. Although cortical astrocytes plated in the presence of poly L-lysine alone form cavities when ECM deposition is disrupted, this effect can be prevented, at least in part, by plating the cells on coverslips coated with both poly L-lysine and either collagen IV or laminin.

Cortical astrocytes were plated on coverslips coated with both poly L-lysine and laminin, and cultured for two days to form a confluent monolayer. Following two days in culture, the cells were treated with TGF-β1 which is a strong inducer of GAG biosynthesis (Asher et al. (2000) Journal of Neuroscience 20: 2427-2438). At the same time, the cells were cultured in the presence of either XT-I antisense oligonucleotide or a control oligonucleotide. Following an additional two days in culture, the cells were fixed in 2% paraformaldehyde, rinsed in PBS, and processed for immunocytochemistry with an antibody that recognizes chondroitin sulfate GAG chains (CS-56; Sigma; goat anti-mouse IgM).

Analysis of CS-56 staining indicated that in the presence of a control antisense oligonucleotide, the astrocytes abundantly express chondroitin sulfate GAG chains following TGF-β1 stimulation, as measured by robust staining with the CS-56 antibody. However, in the presence of XT-I antisense oligonucleotide, CS-56 staining is dramatically reduced.

### Example 5

### Design of XT-I DNA Enzyme

Based on the design of an antisense oligonucleotide that specifically recognizes and inhibits XT-I, we designed an XT-I DNA enzyme. The XT-I DNA enzyme recognizes the same sequence as the XT-I antisense oligonucleotide described in Example 1. The XT-I DNA enzyme comprises a loop sequence, and is thus akin to the 10-23 variety of DNA enzyme described by Santoro and Joyce.

The XT-I DNA enzyme is represented in SEQ ID NO: 33. A control DNA enzyme is provided in SEQ ID NO: 34. One advantage of DNA enzymes over ribozymes or other RNA based agents is that they are DNA based, and thus more stable. However, to further increase the stability and inhibit degradation of the DNA enzymes, the last two nucleotides on both the 5′ and 3′ ends of the XT-I DNA enzyme and the control DNA enzyme were phosphothioated.

FIG. 3 further illustrates the XT-I DNA enzyme. The phosphothioated nucleotides are underlined. The loop sequence characteristic of 10-23 DNA enzymes is shown in bold. This loop sequence does not specifically recognize the target nucleic acid—in this case XT-I.

### Example 6

### Treatment of Cells with a DNA Enzyme Reduces GAG Content

To assess the efficacy of XT-I DNA enzymes, we examined the ability of the XT-I DNA enzyme to decrease GAG content in cultured rat cortical astrocytes. Briefly, cortical astrocytes were cultured for 2 days on poly L-lysine and laminin coated coverslips to form a confluent layer, as described in detail above. These cells were cultured in the presence of TGF-β1 which activates proteoglycan synthesis. After 2 days, these cells were either treated for an additional two days with an XT-I DNA enzyme (a 10-23 DNA enzyme comprising the nucleic acid sequence provided in SEQ ID NO: 33) or a control DNA enzyme (a 10-23 DNA enzyme comprising the nucleic acid sequence provided in SEQ ID NO: 34).

Following two days in the presence of XT-I DNA enzyme or control DNA enzyme, the cultured cells were stained with an antibody which is immunoreactive with chondroitin sulfate GAG chains (CS-56; Sigma). These experiments indicated that in the presence of a control DNA enzyme, the astrocytes abundantly express chondroitin sulfate GAG chains, as measured by robust staining with the CS-56 antibody. However, in the presence of the XT-I DNA enzyme, CS-56 staining is dramatically reduced.

### Example 7

### XT-I Protein is Expressed in Cortical Astrocytes

To further confirm the physiological relevance of the results summarized in Example 6, we examined the expression of XT-I in cultures of rat cortical a strocytes. Cortical astrocytes were cultured for 2 days on poly L-lysine and laminin coated coverslips to form a confluent layer. Expression of XT-I protein was examined using an affinity purified antibody that is immunoreactive with XT-I (prepared by Zymed).

Immunocytochemistry of cortical astrocytes confirmed that cortical astrocytes express XT-I protein. Furthermore, the immunocytochemical analysis indicates that at least one site of expression of XT-I protein in cortical astrocytes is the endoplasmic reticulum (ER).

### Example 8

### XT-I DNA Enzyme Promotes Neuronal Regeneration In Vivo

The foregoing experiments demonstrate that XT-I DNA enzymes decrease GAG content in vitro. To further characterize these DNA enzymes, and to examine their in vivo efficacy, we administered XT-I DNA enzymes to injured mice. Our results indicated that XT-I DNA enzymes work in vivo. Administration of an XT-I DNA enzyme decreases GAG content in vivo. Administration of an XT-I DNA enzyme also promotes neuronal regeneration in vivo following an injury to the spinal cord.

A spinal cord injury was simulated in adult rats using a stab injury of the spinal cord at the C5-C6 level. The dura was opened at the C5-C6 level and a lesion was made by inserting a 25 gauge needle. Following injury, mouse dorsal root ganglia cells (DRGs) were transplanted in the C4-C5 region. The DRGs were derived from adult “green” mice. These mice express GFP under the control of the actin promoter, and thus the DRG cells are easily observable following transplantation.

Following both injury and transplantation of DRG cells, animals were treated, at the cite of injury, with either an XT-IDNA enzyme or a control DNA enzyme. The enzyme was administered via an intrathecally placed PE-10 tubing connected to a P60 tubing filled with the DNA enzymes against XT-I. The tubing was connected to an osmotic minipump (Alzet). After 7 days of administration of the DNA enzyme, the animals were sacrificed and analyzed.

Immunohistochemical anaylsis of sections through the spinal cord of these rats indicated decreased CS-56 staining in XT-I treated animals in comparison to control treated animals. Additional analysis of GFP expression demonstrated that transplanted DRG cells extended processes into and around the injury cite. Analysis of both CS-56 and GFP staining in the same section demonstrated that GAG content is decreased coincident to the regions where transplanted DRG cells migrate and extend neurites.

### Example 9

### XT-I DNA Enzyme Promotes Inter-Mixing of Schwann Cells and Astrocytes

Previous studies have demonstrated that Schwann cells and astrocytes grown in culture do not readily intermingle. When these two cell types are grown in the same culture, each cell type readily grows and migrates until it makes contact with the other cell type. At that point, although the cells continue to grow, they do not intermingle and instead tend to respect the border between the two cell types. This border is characterized by extensive GAG expression. This phenomenon also has in vivo implications for axon growth and regeneration as axon growth is often inhibited at the boundaries between Schwann cell and astrocytes.

Given the ability of XT-I antisense oligonucleotides and XT-I DNA enzymes to decrease GAG content, we assayed the ability of an XT-I DNA enzyme to decrease GAG content in co-cultures of Schwann cells and astrocytes. We further analyzed whether treatment of co-cultures of Schwann cells and astrocytes with an XT-I DNA enzyme promotes inter-mixing of these cell types.

We used a confrontation assay to analyze the degree of cell inter-mixing and GAG expression in the presence versus the absence of an XT-I DNA enzyme. The assay was conducted over approximately 13 days. Schwann cells and astrocytes were plated on a coverslip at some distance from each other. Cells were cultured under the following conditions: in the presence of 8 μM of XT-I DNA enzyme, in the presence of 8 μM mixed based control enzyme, in the presence of 0.2 units/mL chondroitinase ABC, or in the presence of media alone. After approximately 10 days in culture, the two cell types came into contact, and the experiment was then continued for an additional three days. After approximately thirteen days in culture, cells grown under each condition were analyzed to assess the degree of intermixing and to assess GAG content.

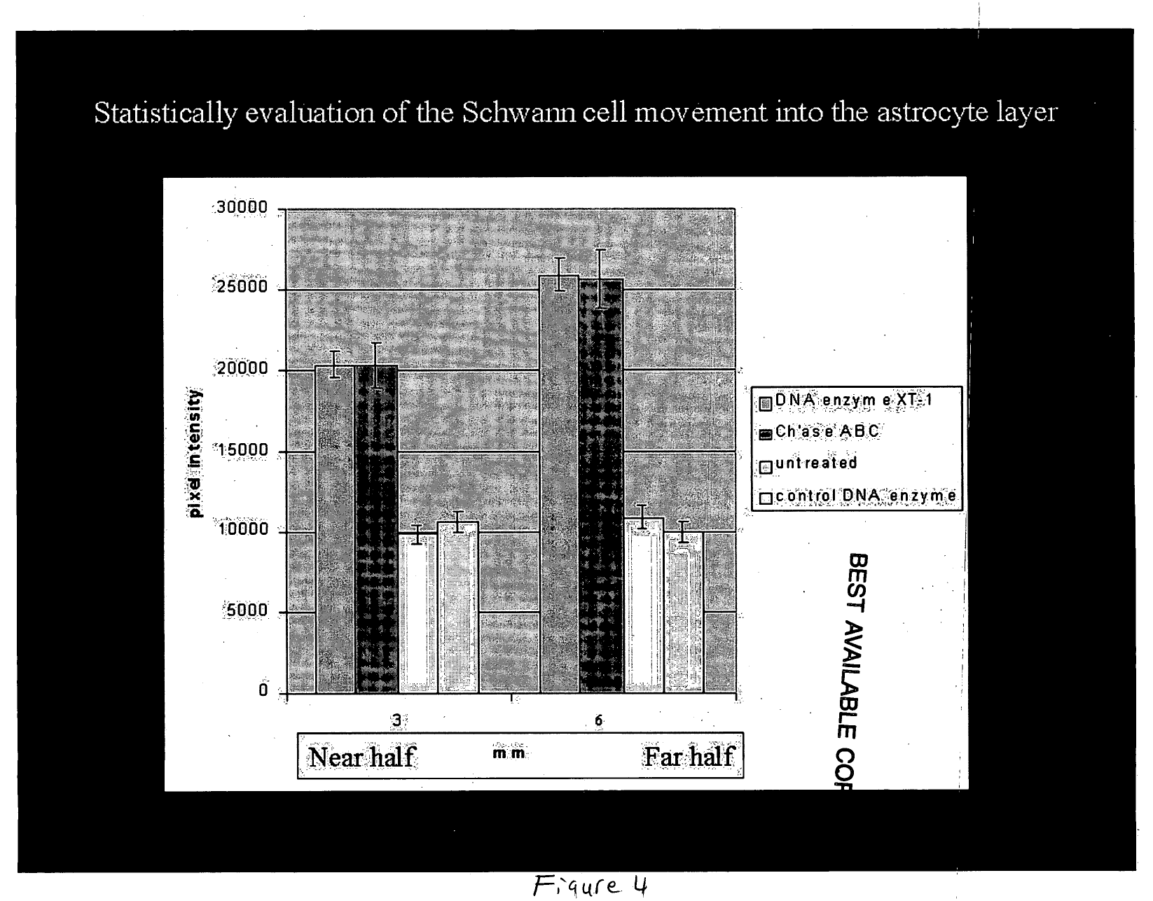

In cells grown in the presence of media alone, very little inter-mixing of Schwann cells and astrocytes was observed. This is consistent with previous observations regarding the behavior of these two cell types. Similarly, little inter-mixing was observed when cells were cultured in the presence of the mixed base control indicating that the presence of a non-specific DNA enzyme alone did not influence cell behavior. In contrast, extensive inter-mixing of Schwann cells and astrocytes was observed in cells grown in the presence of XT-I enzyme. The extent of inter-mixing was greater than that observed in cultures containing chondroitinase ABC—although extensive inter-mixing was also observed under these conditions. These results are summarized graphically in FIG. 4.

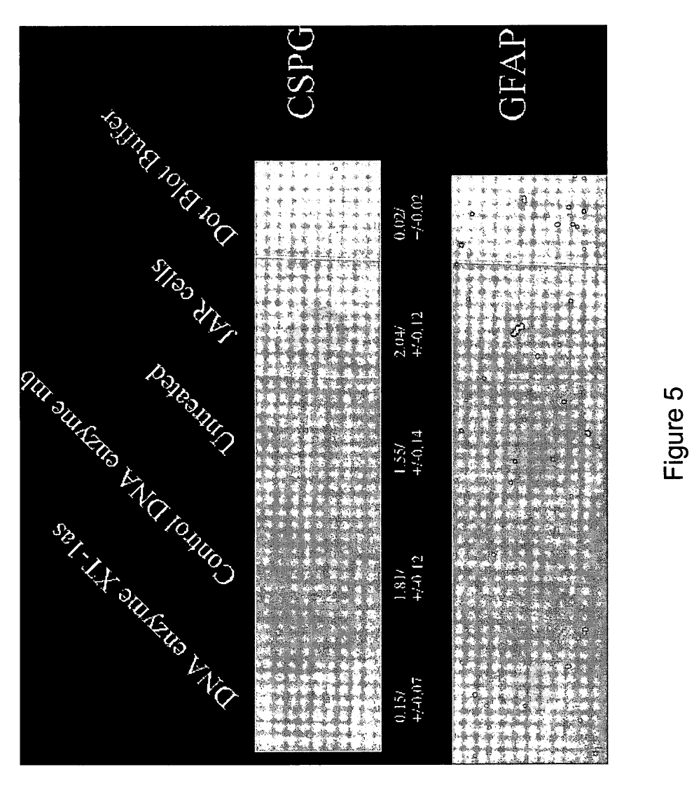

To assess whether this change in cellular behavior correlated with a change in GAG content, we analyzed GAG content in confrontation cultures by Western dot-blot. FIG. 5 summarizes these results which indicated that cells cultured in the presence of XT-I DNA enzyme had a decreased GAG content in comparison to either untreated cells or cells cultured in the presence of the mixed base DNA enzyme control. Taken together, the results summarized in FIGS. 4 and 5 indicated that XT-I DNA enzyme promoted inter-mixing of Schwann cells and astrocytes and decreased GAG content in Schwann cells and astrocytes grown in co-culture.

Briefly, Schwann cells and astrocytes were cultured in confrontation assay, as described above, for a total of 13 days in the presence of XT-I DNA enzyme, control mixed base enzyme, or media alone. After 13 days in culture, cells were harvested to extract protein, and the GAG content in each culture was assessed by dot blot using the CS-56 antibody. Detection of GFAP was used as a loading control to insure analysis of equal concentrations of protein. Protein extracted from JAR cells, which are known to extensively express GAGs, served as a positive control.

### Example 10

### Migration of Dorsal Root Ganglia Cells on Schwann Cells Versus Astrocytes

We assessed the migration of dorsal root ganglia cells (DRGs) plated on either Schwann cells or astrocytes following 12 days of co-culture in the confrontation assay. Schwann cells and astrocytes were cultured for 12 days in the presence of XT-I DNA enzyme, control mixed-based enzyme, chondroitinase ABC, or in media alone. Following 12 days in culture, DRG cells were plated on either Schwann cells or astrocytes within the co-culture, and allowed to attach and migrate for another 2 days.

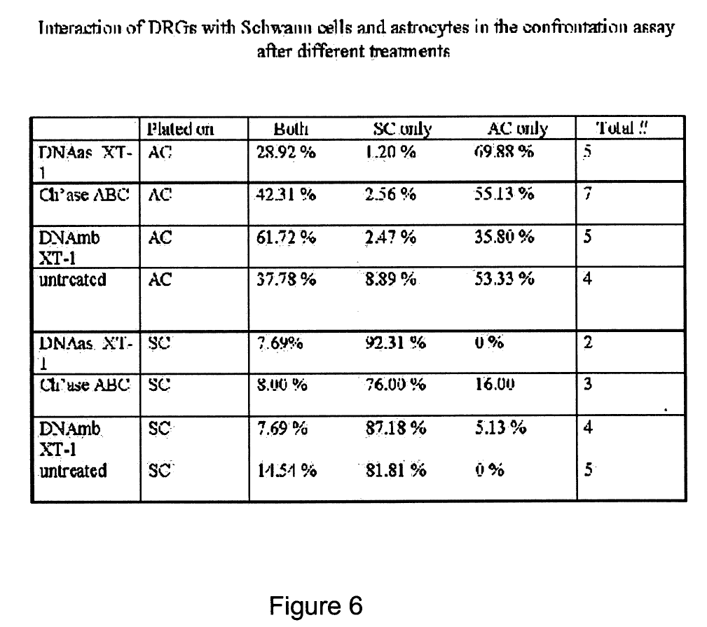

The results of this experiment are summarized in FIG. 6. In this in vitro assay, the culture conditions did not appear to influence of dorsal root ganglia cell movement.

Cell movement was influenced by the identity of the cell type upon which the DRG cell was first plated. When the DRG cell was first plated on Schwann cells (denoted SC in FIG. 6), DRG cell migration respected the border between Schwann cells and astrocytes. However, when the DRG cell was first plated on astrocytes (denoted AC in FIG. 6), DRG cell migration did not necessarily respect the border between the cell types.

Without being bound by theory, the results summarized in FIG. 6 may have the following implications. Firstly, these results indicate that influences from support cells such as Schwann cells and astrocytes may have a significant impact on neuronal cell migration. Secondly, these results may have implications for cellular based treatments for neuronal injuries and diseases. Currently, Schwann cells are sometimes transplanted to sites of neuronal damage to help minimize cavitation that can occur at an injury site.

The cells are thought to act as “bridge cells” to help promote regeneration. However, these results suggest that Schwann cells may not be the optimal candidate “bridge cell”, and that their presence may in fact prevent cell migration across interfaces between neuronal cell types.

### Example 11

### Analysis of Human XT-I Nucleic Acid Sequence

We have analyzed the nucleic acid sequence of human XT-I to identify sequences to which antisense oligonucleotides, DNA enzymes, ribozymes, RNAi constructs, and morpholinos can be designed. Table 1 provides a series of 20′ mers from human XT-1.

TABLE 1

| Human XT-I Nucleotide Sequence | SEQ ID NO |
|---|---|
| ggg aag gtt att caa ccc gc | SEQ ID NO: 41 |
| cag gct caa cgg gac atg ca | SEQ ID NO: 42 |
| ctt ttc ccg tcc agg aaa ta | SEQ ID NO: 43 |
| gtc aat tgc aca ccg gtg ct | SEQ ID NO: 44 |
| caa aac ctt cgt caa ttg ca | SEQ ID NO: 45 |
| agg caa cgc gga tga aac tc | SEQ ID NO: 46 |
| tcc tac ggt acc tct tca ga | SEQ ID NO: 47 |
| cca ggg tat ctc cta cgg ta | SEQ ID NO: 48 |
| caa gtg cga ggt tcc ctc cc | SEQ ID NO: 49 |
| agg gtg ctg acg aaa tcc aa | SEQ ID NO: 50 |
| ttt acg gag cac agt cat aa | SEQ ID NO: 51 |
| cag aac ttc gtt tac gga gc | SEQ ID NO: 52 |
| gca gga tct caa cgg gaa ag | SEQ ID NO: 53 |
| gac cgc gct ccg aag atg gt | SEQ ID NO: 54 |
| caa acc tga tgg ccg gct ca | SEQ ID NO: 55 |
| tgg gtg ccg tca aac ctg at | SEQ ID NO: 56 |
| cca gag cct aaa ccc cgt cc | SEQ ID NO: 57 |
| aac agg cac gga gga acg ca | SEQ ID NO: 58 |
| aaa ttc ctc gtt gcg cct ct | SEQ ID NO: 59 |
| ttg agt cca ctg ccg aat tc | SEQ ID NO: 60 |
| ccg tga ccg tca ttt ggg tg | SEQ ID NO: 61 |
| cat ttg ggt gga tcc cgt ca | SEQ ID NO: 62 |
| ttt ccg agg tcg gca ctg ac | SEQ ID NO: 63 |
| agg aga ggc tat tcc gca ac | SEQ ID NO: 64 |
| aag atc gca agc cca ccc ag | SEQ ID NO: 65 |
| gaa cag ctg ccg ata cta cc | SEQ ID NO: 66 |
| ggc tga gac gtc cct gca ca | SEQ ID NO: 67 |
| gtc ttc gac ggg ctg aga cg | SEQ ID NO: 68 |
| cca ctc ctt tgc ccg cct gg | SEQ ID NO: 69 |
| ggc ctg cgc tcc tac tgg ga | SEQ ID NO: 70 |
| gat gag cct gac ggc atc ca | SEQ ID NO: 71 |
| gaa tgt cta cga tga gcc tg | SEQ ID NO: 72 |
| ctg tac ggg aac tac cct gc | SEQ ID NO: 73 |
| gga cta tta cct gta cgg ga | SEQ ID NO: 74 |
| ttt gcc cgc aag ttt gaa gc | SEQ ID NO: 75 |
| gcc ccc act gcg aca cca tg | SEQ ID NO: 76 |
| cca cag acg atc tgg tga cc | SEQ ID NO: 77 |
| ttc ctg ctg aac cgg agg tt | SEQ ID NO: 78 |
| tgg atg gcg gtt cgg act gg | SEQ ID NO: 79 |
| gag atc ggc gga tcc cag ag | SEQ ID NO: 80 |
| tgg agt gcg acg ctc aca tg | SEQ ID NO: 81 |
| gca agg ttc att cgg aag ca | SEQ ID NO: 82 |
| agt cac acg gcc ggg aca at | SEQ ID NO: 83 |
| tct ccc gat acc gag ata tg | SEQ ID NO: 84 |
| gtg gcg ttt ctc tcc cga ta | SEQ ID NO: 85 |
| gag tgc ggc cga cta ccc ca | SEQ ID NO: 86 |
| gca atg tcc gcg tca ccc cc | SEQ ID NO: 87 |
| acg tgg aca agc gct cta at | SEQ ID NO: 88 |
| ctc tcg gca gtt gca gcg cat g | SEQ ID NO: 89 |
| ggc cgt gcc tct cgg cag tt | SEQ ID NO: 90 |
| agc caa ccc ggt cag aat cg | SEQ ID NO: 91 |
| gac tcc gtg gag tac atg cc | SEQ ID NO: 92 |
| aga acg tgc agt ggg acg ag | SEQ ID NO: 93 |
| ggt tct gcc ccc tcg agg gt | SEQ ID NO: 94 |
| aag gtg act cgg ttc tgc cc | SEQ ID NO: 95 |
| tcc ccc gag acc aag tat ga | SEQ ID NO: 96 |
| agc aac ttc gca ccc agg ac | SEQ ID NO: 97 |
| cgc cac aag tta ggg ctg ct | SEQ ID NO: 98 |
| ctc atc ggc cga aag aga aa | SEQ ID NO: 99 |
| agt ccg ctc atc acc ctg ga | SEQ ID NO: 100 |
| cgc tgg tcg tgt gga att tc | SEQ ID NO: 101 |
| cgg aga acc gcg ggg aca gc | SEQ ID NO: 102 |

The invention contemplates that antisense oligonucleotides, DNA enzymes, ribozymes, RNAi constructs, and morpholino constructs can be designed based on this sequence information. For example, exemplary antisense oligonucleotides, RNAi constructs, or morpholinos comprise a nucleic acid sequence that hybridizes under stringent conditions, including a wash step of 0.2×SSC at 65° C., to a nucleic acid sequence of a XT-I. In one embodiment, the antisense oligonucleotides, RNAi constructs, or morpholinos comprise a nucleic acid sequence that hybridizes under stringent conditions to a nucleic acid sequence represented in any of SEQ ID NOs: 41-102. In another embodiment, the antisense oligonucleotides, RNAi constructs, or morpholinos consist essentially of a nucleic acid sequence that hybridizes under stringent conditions to a nucleic acid sequence represented in any of SEQ ID NOs: 41-102. In still another embodiment, the antisense oligonucleotides, RNAi constructs, or morpholinos consist of a nucleic acid sequence that hybridizes under stringent conditions to a nucleic acid sequence represented in any of SEQ ID NOs: 41-102. In any of the foregoing, exemplary antisense oligonucleotides, RNAi constructs, or morpholinos bind to and inhibit the expression and/or activitiy of XT-I.

Further exemplary antisense oligonucleotides, RNAi constructs, or morpholinos comprise a nucleic acid sequence complementary to a nucleic acid sequence of a XT-I. In one embodiment, the antisense oligonucleotides, RNAi constructs, or morpholinos comprise a nucleic acid sequence complementary to a nucleic acid sequence represented in any of SEQ ID NOs: 41-102. In another embodiment, the antisense oligonucleotides, RNAi constructs, or morpholinos consist essentially of a nucleic acid sequence complementary to a nucleic acid sequence represented in any of SEQ ID NOs: 41-102. In still another embodiment, the antisense oligonucleotides, RNAi constructs, or morpholinos consist of a nucleic acid sequence complementary to a nucleic acid sequence represented in any of SEQ ID NOs: 41-102. In any of the foregoing, exemplary antisense oligonucleotides, RNAi constructs, or morpholinos bind to and inhibit the expression and/or activitiy of XT-I.

To further illustrate, exemplary DNA enzymes comprise a nucleic acid sequence that hybridizes under stringent conditions, including a wash step of 0.2×SSC at 65° C., to a nucleic acid sequence of a XT-I. In one embodiment, the DNA enzymes comprise a nucleic acid sequence that hybridizes under stringent conditions to a nucleic acid sequence represented in any of SEQ ID NOs: 41-102. In another embodiment, the DNA enzymes consist essentially of a nucleic acid sequence that hybridizes under stringent conditions to a nucleic acid sequence represented in any of SEQ ID NOs: 41-102. In still another embodiment, the DNA enzymes consist of a nucleic acid sequence that hybridizes under stringent conditions to a nucleic acid sequence represented in any of SEQ ID NOs: 41-102. In any of the foregoing, exemplary DNA enzymes bind to and inhibit the expression and/or activitiy of XT-I.

Further exemplary DNA enzymes comprise a nucleic acid sequence complementary to a nucleic acid sequence of a XT-I. In one embodiment, the DNA enzymes comprise a nucleic acid sequence complementary to a nucleic acid sequence represented in any of SEQ ID NOs: 41-102. In another embodiment, the DNA enzymes consist essentially of a nucleic acid sequence complementary to a nucleic acid sequence represented in any of SEQ ID NOs: 41-102. In still another embodiment, the DNA enzymes consist of a nucleic acid sequence complementary to a nucleic acid sequence represented in any of SEQ ID NOs: 41-102.

In any of the foregoing, exemplary DNA enzymes bind to and inhibit the expression and/or activitiy of XT-I. We note, however, that DNA enzymes contain a sequence interspersed between sequences that bind to the nucleotide sequence of the xylotransferase. Thus, a DNA enzyme that binds to and inhibits the expression and/or activitiy of a xylotransferase can be represented by the following general formula:

B₁-X-B₂

wherein X corresponds to a DNA enzyme nucleotide sequence, B₁ corresponds to a nucleotide sequence complementary to a nucleotide sequence of a xylotransferase, and B₂ corresponds to a nucleotide sequence complementary to a nucleotide sequence of a xylotransferase, and wherein B₁ and B₂ are complementary to nucleotide sequences of a xylotransferase that are adjacent to but separated by at least one nucleotide.

In one embodiment, the xylotransferase is XT-I. In one embodiment, B₁ and B₂ are separated by at least 2, 3, 4, or 5 nucleotides.

Any of the foregoing inhibitory agents can be formulated as compositions and pharmaceutical compositions, and one of skill in the art can readily test these agents in any of the in vitro or in vivo assays provided herein to select agents that inhibit the expression and/or activity of XT-I.

### Example 12

### Analysis of Human XT-II Nucleic Acid Sequence

We have analyzed the nucleic acid sequence of human XT-II to identify sequences to which antisense oligonucleotides, DNA enzymes, ribozymes, RNAi constructs, and morpholinos can be designed. Table 2 provides a series of 20′ mers from human XT-II.

TABLE 2

| Human XT-II Nucleotide Sequence | SEQ ID NO |
|---|---|
| agg ccc tgc gct cga ggc ct | SEQ ID NO: 103 |
| ttc cag ggc ctg agt agc at | SEQ ID NO: 104 |
| cca ccc cac aac gag tac at | SEQ ID NO: 105 |
| gtc aaa gca gac ggg cga ct | SEQ ID NO: 106 |
| tcc ccc gac ccc aaa tca ga | SEQ ID NO: 107 |
| acc tgt acg gca gct acc cc | SEQ ID NO: 108 |
| tga taa cgt gcg ggt gac gc | SEQ ID NO: 109 |
| cgg gtg acg ccc tgg cgc at | SEQ ID NO: 110 |
| cct ggc gca tgg tta cca tc | SEQ ID NO: 111 |
| cag atc cca gca ggc att gt | SEQ ID NO: 112 |
| ggg cga gcg gca gat ccc ag | SEQ ID NO: 113 |
| gac tca cac atg tgg cgc ct | SEQ ID NO: 114 |
| cca tga gtg cga ctc aca ca | SEQ ID NO: 115 |
| acc ggc tct tcc atg agt gc | SEQ ID NO: 116 |
| ggc cgg gac aac tcc agg tt | SEQ ID NO: 117 |
| aac cag gac caa tga gga gc | SEQ ID NO: 118 |
| ctg act atc caa cca gga cc | SEQ ID NO: 119 |
| tgc ggg acc tgc tag agg tg | SEQ ID NO: 120 |
| ctg cgg agc atg cgg gac ct | SEQ ID NO: 121 |
| gac gat gta cct gcg gag ca | SEQ ID NO: 122 |
| cca gcc tcc tga cga tgt ac | SEQ ID NO: 123 |
| ggt tcg tgc tga cac gca gc | SEQ ID NO: 124 |
| ggt gga tgg cgg ttc tga ct | SEQ ID NO: 125 |
| acc cca ctc tgc agg ttt ga | SEQ ID NO: 126 |
| ccc aat ggg cac ccc act ct | SEQ ID NO: 127 |
| gag aac acc tac gac gcg gc | SEQ ID NO: 128 |
| ccc ggc acg cca gcc ctc aa | SEQ ID NO: 129 |
| ctg gac ttc cac ctg tac gg | SEQ ID NO: 130 |
| tct tcg ccc gga agt tcg ag | SEQ ID NO: 131 |
| gtt ggc act gat tgg gac cc | SEQ ID NO: 132 |
| tga agc tgt tgg ggc gca gt | SEQ ID NO: 133 |
| caa ggg tcg ctg aag ctg tt | SEQ ID NO: 134 |
| gct gat gcc cca agg gtc gc | SEQ ID NO: 135 |
| gca gag acg ctt gag atg tg | SEQ ID NO: 136 |
| ccg ctg gac gag cct gtg gc | SEQ ID NO: 137 |
| caa aga gcg tct ttt ccg ga | SEQ ID NO: 138 |
| cca ggg gcc ggc aga gac gc | SEQ ID NO: 139 |
| gag act gag gtc acg caa ta | SEQ ID NO: 140 |
| agt aga tac gga gac tga gg | SEQ ID NO: 141 |
| atg aca tca cag tag ata cg | SEQ ID NO: 142 |
| gcc aca tct tat gac atc ac | SEQ ID NO: 143 |
| tcg acc caa cct atg tgg tg | SEQ ID NO: 144 |
| gtg gtc tgg atc gac cca ac | SEQ ID NO: 145 |
| ttg acc ttc aac cgc aaa ct | SEQ ID NO: 146 |
| ctg gga acc gct ggg tga ga | SEQ ID NO: 147 |
| aga ccc acc ttc ttc gcc cg | SEQ ID NO: 148 |
| tgc tcc cag ccg agt cct tc | SEQ ID NO: 149 |
| cac aga tga ccc gct tgt gg | SEQ ID NO: 150 |
| ccc agg gct atg ata acg tg | SEQ ID NO: 151 |
| gca gta acc agc cgg cag ag | SEQ ID NO: 152 |
| agc cag ccg gcg ggt ccc ac | SEQ ID NO: 153 |
| ctg ccc cac ctc cgg aag cc | SEQ ID NO: 154 |
| tcc gga agc ccc agg ccg cc | SEQ ID NO: 155 |
| ccc agc cca cgg aca atg gc | SEQ ID NO: 156 |
| agg gag cgt gga ggg cgc cc | SEQ ID NO: 157 |
| acg gag ata cag gga gcg tg | SEQ ID NO: 158 |
| ttc cca cca cac gga gat ac | SEQ ID NO: 159 |
| gac gca ctg tct gca ctg gc | SEQ ID NO: 160 |
| agt gcg aga tcg tgg gca ag | SEQ ID NO: 161 |
| ttc acc ccc aag tgc gag at | SEQ ID NO: 162 |
| tgg gag cct cat gcc caa gg | SEQ ID NO: 163 |
| cgg cat cca gtg gga tga ga | SEQ ID NO: 164 |
| cgg tgc gaa tcg cct aca tg | SEQ ID NO: 165 |
| gat ggc ccc ccg gtg cga at | SEQ ID NO: 166 |
| ctg aag cgt ctc ctc aag gc | SEQ ID NO: 167 |
| tct ttt aca tcc atg tgg ac | SEQ ID NO: 168 |
| cgt tta tca cga gca gca ct | SEQ ID NO: 169 |
| tcc tca agg ccg ttt atc ac | SEQ ID NO: 170 |
| aag cgt tcc gac tac ctg ca | SEQ ID NO: 171 |
| ggt ggt acg ggc agt aac ca | SEQ ID NO: 172 |
| acg agg cgg gcg aga aag ga | SEQ ID NO: 173 |
| gca ggg cga cgg ggc agc ac | SEQ ID NO: 174 |
| cca gga gtg ccc gtg gcc aa | SEQ ID NO: 175 |
| ggc gaa ggt tcc aag gac ac | SEQ ID NO: 176 |
| cgc gcg agt gca gaa gct gg | SEQ ID NO: 177 |
| ccg tgg cca agg tgg tac gg | SEQ ID NO: 178 |
| ccc gcc tcc cta ggc gtg ga | SEQ ID NO: 179 |
| aag cgt tcc gac tac ctg ca | SEQ ID NO: 180 |
| ggt ggt acg ggc agt aac ca | SEQ ID NO: 181 |
| gcg gcc ggg ttg cag ggc tg | SEQ ID NO: 182 |
| agt acc cgt gga gga ccc gg | SEQ ID NO: 183 |
| ggc ccc agc cag tac ccg tg | SEQ ID NO: 184 |
| gag gac ccg gga aat tgc ac | SEQ ID NO: 185 |
| agg tgg aca cag tat gaa ct | SEQ ID NO: 186 |
| acg cga gaa ggg cac cag ca | SEQ ID NO: 187 |
| gaa gat gtg caa tag ggc tc | SEQ ID NO: 188 |

The invention contemplates that antisense oligonucleotides, DNA enzymes, ribozymes, RNAi constructs, and morpholino constructs can be designed based on this sequence information. For example, exemplary antisense oligonucleotides, RNAi constructs, or morpholinos comprise a nucleic acid sequence that hybridizes under stringent conditions, including a wash step of 0.2×SSC at 65° C., to a nucleic acid sequence of a XT-II. In one embodiment, the antisense oligonucleotides, RNAi constructs, or morpholinos comprise a nucleic acid sequence that hybridizes under stringent conditions to a nucleic acid sequence represented in any of SEQ ID NOs: 103-188. In another embodiment, the antisense oligonucleotides, RNAi constructs, or morpholinos consist essentially of a nucleic acid sequence that hybridizes under stringent conditions to a nucleic acid sequence represented in any of SEQ ID NOs: 103-188. In still another embodiment, the antisense oligonucleotides, RNAi constructs, or morpholinos consist of a nucleic acid sequence that hybridizes under stringent conditions to a nucleic acid sequence represented in any of SEQ ID NOs: 103-188. In any of the foregoing, exemplary antisense oligonucleotides, RNAi constructs, or morpholinos bind to and inhibit the expression and/or activitiy of XT-II.

Further exemplary antisense oligonucleotides, RNAi constructs, or morpholinos comprise a nucleic acid sequence complementary to a nucleic acid sequence of a XT-II. In one embodiment, the antisense oligonucleotides, RNAi constructs, or morpholinos comprise a nucleic acid sequence complementary to a nucleic acid sequence represented in any of SEQ ID NOs: 103-188. In another embodiment, the antisense oligonucleotides, RNAi constructs, or morpholinos consist essentially of a nucleic acid sequence complementary to a nucleic acid sequence represented in any of SEQ ID NOs: 103-188. In still another embodiment, the antisense oligonucleotides, RNAi constructs, or morpholinos consist of a nucleic acid sequence complementary to a nucleic acid sequence represented in any of SEQ ID NOs: 103-188. In any of the foregoing, exemplary antisense oligonucleotides, RNAi constructs, or morpholinos bind to and inhibit the expression and/or activitiy of XT-II.

To further illustrate, exemplary DNA enzymes comprise a nucleic acid sequence that hybridizes under stringent conditions, including a wash step of 0.2×SSC at 65° C., to a nucleic acid sequence of a XT-II. In one embodiment, the DNA enzymes comprise a nucleic acid sequence that hybridizes under stringent conditions to a nucleic acid sequence represented in any of SEQ ID NOs: 103-188. In another embodiment, the DNA enzymes consist essentially of a nucleic acid sequence that hybridizes under stringent conditions to a nucleic acid sequence represented in any of SEQ ID NOs: 103-188. In still another embodiment, the DNA enzymes consist of a nucleic acid sequence that hybridizes under stringent conditions to a nucleic acid sequence represented in any of SEQ ID NOs: 103-188. In any of the foregoing, exemplary DNA enzymes bind to and inhibit the expression and/or activitiy of XT-II.

Further exemplary DNA enzymes comprise a nucleic acid sequence complementary to a nucleic acid sequence of a XT-II. In one embodiment, the DNA enzymes comprise a nucleic acid sequence complementary to a nucleic acid sequence represented in any of SEQ ID NOs: 103-188. In another embodiment, the DNA enzymes consist essentially of a nucleic acid sequence complementary to a nucleic acid sequence represented in any of SEQ ID NOs: 103-188. In still another embodiment, the DNA enzymes consist of a nucleic acid sequence complementary to a nucleic acid sequence represented in any of SEQ ID NOs: 103-188.

In any of the foregoing, exemplary DNA enzymes bind to and inhibit the expression and/or activitiy of XT-II. We note, however, that DNA enzymes contain a sequence interspersed between sequences that bind to the nucleotide sequence of the xylotransferase. Thus, a DNA enzyme that binds to and inhibits the expression and/or activitiy of a xylotransferase can be represented by the following general formula:

B₁-X-B₂

wherein X corresponds to a DNA enzyme nucleotide sequence, B₁ corresponds to a nucleotide sequence complementary to a nucleotide sequence of a xylotransferase, and B₂ corresponds to a nucleotide sequence complementary to a nucleotide sequence of a xylotransferase, and wherein B₁ and B₂ are complementary to nucleotide sequences of a xylotransferase that are adjacent to but separated by at least one nucleotide.

In one embodiment, the xylotransferase is XT-II. In one embodiment, B 1 and B2 are separated by at least 2, 3, 4, or 5 nucleotides.

Any of the foregoing inhibitory agents can be formulated as compositions and pharmaceutical compositions, and one of skill in the art can readily test these agents in any of the in vitro or in vivo assays provided herein to select agents that inhibit the expression and/or activity of XT-II.

### Example 13

### Human XT-I DNA Enzyme

As outlined in detail above, exemplary antisense oligonucleotides, DNA enzymes, ribozymes, RNAi constructs, and morpholinos that bind to and inhibit the expression and/or activitiy of XT-I can be made based on, for example, the sequences outlined in Table 1. As further proof of this contention, we have designed a DNA enzyme that binds to and inhibits the expression and/or activity of human XT-I and decreased GAG content.

We designed a DNA enzyme based on the human XT-I sequence depicted in SEQ ID NO: 87. Briefly, a region of the DNA enzyme is complementary to SEQ ID NO: 87, and the DNA enzyme will hybridize under stringent conditions to a nucleic acid containing SEQ ID NO: 87.

The particular DNA enzyme is represented in the following: ggg ggt gac ggg cta gct aca acg agg aca ttg c (SEQ ID NO: 39). Note that this DNA enzyme can be represented in the general formula B₁-X-B₂, as discussed in detail above, and that B₁ and B₂ correspond to sequences in XT-I that are adjacent but separated by one nucleotide.

### Example 14

### Human XT-I DNA Enzyme Reduce GAG Content In vitro

As detailed in example 13, we designed a DNA enzyme based on the human XT-I sequence provided in SEQ ID NO: 87. The DNA enzyme is provided in SEQ ID NO: 39. To test the efficacy of this DNA enzyme, we obtained human astrocytes (ScienCell Research Laboratories). Human astrocytes were plated on poly L-lysine/laminin coated glass coverslips in 24 well plates at a concentration of approximately 40,000 cells/well. Cells were treated for 2-3 days with either media containing the human DNA enzyme represented in SEQ ID NO: 39, or with media containing a control DNA enzyme. The control DNA enzyme has the following sequence: tagctgtccagcaacatcgatcggcgtcacaatt (SEQ ID NO: 40).

Following 2-3 days of treatment, the astrocytes were fixed in 2% PFA for 30 minutes, followed by fixing in 4% PFA for 30 minutes, and then stained to assess proteoglycan content. Treatment with a human XT-I DNA enzyme decreased GAG content in the human astrocytes.

Additional References

WO99/08533 U.S. Pat. No. 6,110,462

WO01/49831 U.S. Pat. No. 5,807,718

Stichel and Muller (1998) Cell Tissue Research 294: 1-9.

Qiu et al. (2000) Glia 29: 166-174.

Margolis and Margolis (1997) Cell Tissue Research 290: 343-348.

Davies et al. (1997) Nature 390: 680-683.

Grumet et al. (1996) Perspect. Dev. Neurobiol. 3: 319-330.

Fidler et al. (1999) J. Neurosci. 19: 8778-8788.

Hoffman-Kim et al. (1998) J Neurosci. 18: 5881-5890.

Kearns et al. (1991) Biochemistry 30: 7477-7483.

Martin et al. (1999) Plant Physiology 120: 553-558.

Sun et al. (1999) Journal of Biological Chemistry 274: 17236-17241.

Wilson (2002) Journal of Biological Chemistry 277: 21207-21212.

Gotting et al. (2002) Clin Chim Acta 317: 199-202.

Koslowski et al. (2001) European Respir J 18: 347-356.

Gotting et al. (2000) Journal of Molecular Biology 304: 517-528.

Kuhn et al. (2001) Journal of Biological Chemistry 276: 4940-4947.

Pfeil and Wenzel (2000) Glycobiology 10: 803-807.

Gotting et al. (2000) Acta Derm Venereol 80: 60-61.

Gotting et al. (1999) Journal of Invest Dermatol 112: 919-924.

Bai et al. (1999) Journal of Biological Chemistry 274: 13017-13024.

Gotting et al. (1998) Journal of Protein Chemistry 17: 295-302.

Brinkmann et al. (1997) Journal of Biological Chemistry 272: 11171-11175.

Weilke et al. (1997) Clin Chem 43: 45-51.

Asher et al. (2000) Journal of Neuroscience 20: 2427-2438.

McKeon et al. (1995) Experimental Neurology 136: 32-43.

McKeon et al. (1999) Journal of Neuroscience 19: 10778-10788.

Garwood et al. (1999) Journal of Neuroscience 19: 3888-3899.

Grumet et al. (1993) Journal of Cell Biology 120: 815-824.

Haas et al. (1999) Journal of Neuroscience 19: 9953-9963.

Lee et al. (2000) Glia 30: 105-121.

Maurel et al. (1994) PNAS91: 2512-2516.

Pindzola et al. (1993) Developmental Biology 156: 34-48.

Santoro and Joyce (1997) PNAS 94: 4262-4266.

Snow et al. (1990) Experimental Neurology 109: 111-130.

Uyama et al. (2002) Journal of Biological Chemistry 277: 8841-8846.

Uchimura et al. (2002) Journal of Biological Chemistry 277: 1443-1450.

Tsuchida et al. (1999) European Journal of Biochemistry 264: 461-467.

Rohrmann et al. (1985) European Journal of Biochemistry 148: 463-469

Tone et al. (1999) FEBS Letter 459: 415-420.

Kitagawa et al. (1998) Journal of Biological Chemistry 273: 6615-6618.

Lakatos et al. (2000) Glia 32: 214-225.

Takami et al. (2002) Journal of Neuroscience 22: 6670-6681.

Cheng and Zochodne. (2002) Neuroscience 115: 321-329.

Bunge. (2002) Progress in Brain Research 137: 275-282.

Bamber et al. (2001) European Journal of Neuroscience 13: 257-268.

Benn et al. (2002) Neuron 36: 45-56.

Borisoff et al. (2003) Molecular Cellular Neuroscience 22: 405-416.

Bunge and Kleitman. (1999) Neurotrophins and neuroprotection improve axonal regeneration into Schwann cell transplants placed in transected adult rat spinal cord. In: CNS regeneration: basic science and clinical aspects (Tuszinski M. H., Kordower J., eds) pp 631-646. San Diego: Academic.

Cai et al. (2002) Neuron 35: 711-719.

Carlstedt. (1997) Journal Anatomy 190: 51-56.

Chen et al. (2000) Nature 403: 434-439.

Chong et al. (1996) Journal of Comparative Neurology 370: 97-104.

Chong et al. (1999) Journal of Comparative Neurology 410: 42-54.

Condic. (2001) Journal of Neuroscience 21: 4782-4788.

Davies et al. (1999) Journal of Neuroscience 19: 5810-5822.

Dergham et al. (2002) Journal of Neuroscience 22: 6570-6577.

Eddlestone and Mucke. (1993) Neuroscience 54: 15-36.

Fitch et al. (1999) Journal of Neuroscience 19: 8182-8198.

Ghirnikar and Eng. (1995) Glia 14: 145-152.

Golding et al. (1999) Glia 26: 309-323.

Golding et al. (1996) Molecular Cellular Neuroscience 7: 191-203.

Hoffmann et al. (1984) Connective Tissue Research 12: 151-163.

Kozlova et al. (1995) NeuroReport 6: 269-272.

Lal et al. (1996) Mol Chem Neuropathol 29: 93-104.

Liuzzi and Lasek. (1987) Science 237: 642-645.

Menei et al. (1998) European Journal of Neuroscience 10: 607-621.

Neumann and Woolf. (1999) Neuron 23: 83-91.

Neumann et al. (2002) Neuron 34: 109-117.

Nomura et al. (2002) Brain Research 950: 21-30.

Oudega and Hagg. (1999) Brain Research 818: 431-438.

Pindzola et al. (1993) Developmental Biology 156: 34-48.

Qiu et al. (2002). Neuron 34: 895-903.

Ramer et al. (2002) Molecular Cellular Neuroscience 19: 239-249.

Ramon-Cueto and Nieto-Sampedro. (1994) Experimental Neurology 127: 232-244.

Romero et al. (2001) Journal of Neuroscience 21: 8408-8416.

Snow et al. (2002) Journal of Neurobiology 51: 285-301.

Zhang et al. (2001). Mol Cell Neurosci 17: 444-459.

Zhang et al. (2000) Mol Cell Neurosci 16: 71-86.

Bradbury et al. (2002) Nature 416: 636-640.

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of reducing GAG content in a glial scar comprising inhibiting one or more of the following:
inhibiting the expression of primary proteoglycans;
inhibiting the expression and/or activity of a chain initiation enzyme; and
inhibiting the expression and/or activity of a chain elongation enzyme.

2. The method of claim 1-, wherein the primary proteoglycans are selected from the group consisting of neurocan, NG2, and phosphocan.

3. The method of claim 1; wherein the chain initiation enzyme is a xylotransferase.

4. The method of claim 1, wherein the chain elongation enzyme is selected from the group consisting of N-acetylgalactosaminyl transferase, glucuronosyltransferase, glucosaminyltransferase, galactosaminyltransferase, N-sulfotransferase, 6-sulfotransferase, 3-sulfotransferase, 1,4-glucosaminyltransferase, 1,4-galactosaminyltransferase, N-acetylglucosamine, and glucuronic acid.

5. The method of any one of claims 1 or 2, wherein expression of the primary proteoglycan is inhibited by administering an agent.

6. The method of claim 5, wherein said agent is selected from the group consisting of antisense oligonucleotides that bind a nucleic acid sequence encoding a proteoglycan; ribozymes, DNA enzymes, RNAi constructs, and small molecules.

7. The method of claim 6, wherein the antisense oligonucleotide binds a nucleic acid as set forth in any one of SEQ ID No: 17, SEQ ID No: 19, SEQ ID No: 21, SEQ ID No: 23, SEQ ID No: 25, SEQ ID No: 27, SEQ ID No: 29, and SEQ ID No: 31.

8. The method of any one of claims 1 or 3, wherein expression and/or activity of the chain initiation enzyme is inhibited by administering an agent.

9. The method of claim 8, wherein the agent is selected from the group consisting of antagonists, antibodies, antisense oligonucleotides that bind a nucleic acid sequence encoding a chain initiation enzyme; ribozymes, DNA enzymes, RNAi constructs, and small molecules.

10. The method of claim 9, wherein the antisense oligonucleotide binds a nucleic acid as set forth in any one of SEQ ID NO: 1, SEQ ID NO: 3, SEQ ID NO: 5, SEQ ID NO: 7, SEQ ID NO: 9, and SEQ ID NO: 11.

11. The method of claim 8, wherein the antisense oligonucleotides are selected from the group consisting of SEQ ID NO: 37 and SEQ ID NO: 38.

12. The method of claim 8, wherein the agent is a DNA enzyme.

13. The method of claim 12, wherein the DNA enzyme is set forth in SEQ ID NO: 33 or SEQ ID NO: 39.

14. The method of any one of claims 1 or 4, wherein expression and/or activity of the chain elongation enzyme is inhibited by administering an agent.

15. The method of claim 14, wherein the agent is selected from the group consisting of antagonists, antibodies, antisense oligonucleotides that bind a nucleic acid sequence encoding a chain initiation enzyme; ribozymes, DNA enzymes, RNAi constructs, and small molecules.

16. The method of claim 15, wherein the antisense oligonucleotide binds a nucleic acid as set forth in any one of SEQ ID No: 13 or SEQ ID No: 15.

17. A method of promoting neuronal regeneration comprising inhibiting a chain initiation enzyme.

18. The method of claim 17, wherein the chain initiation enzyme is a xylotransferase.

19. The method of claim 18, wherein the enzyme is inhibited by administering an agent.

20. The method of claim 19, wherein the agent is selected from the group consisting of antagonists, antibodies, antisense oligonucleotides that bind a nucleic acid sequence encoding a chain initiation enzyme, ribozymes, DNA enzymes, RNAi constructs, and small molecules.

21. The method of claim 20, wherein the antisense oligonucleotide binds a nucleic acid as set forth in any one of SEQ ID NO: 1, SEQ ID NO: 3, SEQ ID NO: 5, SEQ ID NO: 7, SEQ ID NO: 9, and SEQ ID NO: 11.

22. The method of claim 20, wherein the antisense oligonucleotides are selected from the group consisting of SEQ ID NO: 37 and SEQ ID NO: 38.

23. The method of claim 20, wherein the agent is a DNA enzyme.

24. The method of claim 23, wherein the DNA enzyme is set forth in SEQ ID NO: 33 or SEQ ID NO: 39.

25. The method of claim 17, further comprising administering a growth factor or a neurotrophic factor.

26. The method of claim 25, wherein the neurotropic factor is selected from the group consisting of nerve growth factor, brain-derived growth factor, neurotrophin 3, neurotrophin 4, neurotrophin 5, glial derived neurotrophic factor, and ciliary neurotrophic factor.

27. The method of claim 25, wherein the growth factor is basic fibroblast growth factor.

28. The method of claim 26 or 27, further comprising administering a proteoglycan specific enzyme.

29. A method of screening to identify and/or characterize an agent, wherein said agent is capable of one or more of the following:
(i) inhibiting the expression of a primary proteoglycan;
(ii) inhibiting the expression and/or activity of a chain initiation enzyme;
(iii) inhibiting the expression and/or activity of a chain elongation enzyme; or
(iv) inhibiting the expression and/or activity of a chain sulfation enzyme.

30. The method of claim 29, wherein said agent promotes neuronal regeneration and/or promotes the inter-mixing of Schwann cells and astrocytes.

31. A method of screening to identify and/or characterize an agent, wherein said agent is capable of one or more of the following:
(i) reducing scar formation;
(ii) promoting inter-mixing of Schwann cells and astrocytes; or
(iii) promoting neuronal regeneration.

32. The method of any of claims 29-31, wherein said agent is selected from the group consisting of antagonists, antibodies, antisense oligonucleotides, ribozymes, DNA enzymes, RNAi constructs, and small organic molecules.

33. The method of claim 32, wherein said method comprises screening a library of agents.

34. The method of claim 29 or 31, further comprising formulating a pharmaceutical preparation of an agent identified and/or characterized by said method and a pharmaceutically acceptable carrier or excipient.

35. A pharmaceutical preparation comprising an agent identified by the method of claim 29 or 31 and a pharmaceutically acceptable carrier or excipient.

36. The method of claim 34, further comprising packaging, marketing, and selling said pharmaceutical preparation.

37. A kit comprising the pharmaceutical preparation of claim 35 and instructions for the use of said pharmaceutical preparation in human or non-human patients.

38. Use of an agent in the manufacture of a medicament for decreasing expression and/or activity of a xylotransferase, wherein said agent is a DNA enzyme that binds to and inhibits expression and/or activity of a xylotransferase.

39. Use of an agent in the manufacture of a medicament for decreasing expression and/or activity of a xylotransferase, wherein said agent is an antisense oligonucleotide that binds to and inhibits expression and/or activity of a xylotransferase.

40. A composition comprising an agent, wherein said agent inhibits the expression and/or activity of a xylotransferase, and wherein said agent is a DNA enzyme that binds to and inhibts the expression and/or activity of a xylotransferase.

41. A composition comprising an agent, wherein said agent inhibits the expression and/or activity of a xylotransferase, and wherein said agent is an antisense oligonucleotide that binds to and inhibts the expression and/or activity of a xylotransferase.

42. A composition comprising an agent, wherein said agent inhibits the expression and/or activity of a xylotransferase, and wherein said agent is an RNAi construct that binds to and inhibts the expression and/or activity of a xylotransferase.

43. The composition of any of claims 40-42, wherein the xylotransferase is XT-I.

44. The composition of any of claims 40-42, wherein the xylotransferase is XT-II.

45. The composition of any of claims 40-42, wherein the xylotransferase is XT-I and XT-II.

46. A DNA enzyme as set forth in SEQ ID No: 33 or SEQ ID NO: 39.

47. An antisense oligonucleotide as set forth in any one of SEQ ID No: 37 or SEQ ID No: 38.

48. A composition comprising a DNA enzyme, wherein said DNA enzyme binds to and inhibits the expression and/or activitiy of a xylotransferase, wherein said DNA enzyme is represented by the general formula
B₁-X-B₂
wherein X corresponds to a DNA enzyme nucleotide sequence, B₁ corresponds to a nucleotide sequence complementary to a nucleotide sequence of a xylotransferase, and B₂ corresponds to a nucleotide sequence complementary to a nucleotide sequence of a xylotransferase, and wherein B₁ and B₂ are complementary to nucleotide sequences of a xylotransferase that are adjacent but separated by at least one nucleotide.

49. The composition of claim 48, wherein the xylotransferase is XT-I

50. The composition of 48, wherein the xylotransferase is XT-II.

51. The composition of claim 48, wherein the xylotransferase is XT-I and XT-II.

52. The composition of claim 48, wherein said composition is a pharmaceutical composition formulated in a pharmaceutically acceptable carrier.

53. Use of an agent in the manufacture of a medicament for decreasing GAG content, wherein said agent is a DNA enzyme that binds to and inhibits expression and/or activity of a xylotransferase.

54. Use of an agent in the manufacture of a medicament for decreasing GAG content, wherein said agent is an antisense oligonucleotide that binds to and inhibits expression and/or activity of a xylotransferase.
